SEQUENCE LISTING (16 sequences)

SEQ ID NO: 1 - DNA, 1254 bp, Mus musculus
agagacggccgcatcttcttgtgcagtgccagcctcgtcccgtagacaaaatggtgaaggtcggtgtgaacggatttggccgtattgggcgcctggtcaccagggctgccatttgcagtggcaaagtggagattgttgccatcaacgaccccttcattgacctcaactacatggtctacatgttccagtatgactccactcacggcaaattcaacggcacagtcaaggccgagaatgggaagcttgtcatcaacgggaagcccatcaccatcttccaggagcgagaccccactaacatcaaatggggtgaggccggtgctgagtatgtcgtggagtctactggtgtcttcaccaccatggagaaggccggggcccacttgaagggtggagccaaaagggtcatcatctccgccccttctgccgatgcccccatgtttgtgatgggtgtgaaccacgagaaatatgacaactcactcaagattgtcagcaatgcatcctgcaccaccaactgcttagcccccctggccaaggtcatccatgacaactttggcattgtggaagggctcatgaccacagtccatgccatcactgccacccagaagactgtggatggcccctctggaaagctgtggcgtgatggccgtggggctgcccagaacatcatccctgcatccactggtgctgccaaggctgtgggcaaggtcatcccagagctgaacgggaagctcactggcatggccttccgtgttcctacccccaatgtgtccgtcgtggatctgacgtgccgcctggagaaacctgccaagtatgatgacatcaagaaggtggtgaagcaggcatctgagggcccactgaagggcatcttgggctacactgaggaccaggttgtctcctgcgacttcaacagcaactcccactcttccaccttcgatgccggggctggcattgctctcaatgacaactttgtcaagctcatttcctggtatgacaatgaatacggctacagcaacagggtggtggacctcatggcctacatggcctccaaggagtaagaaaccctggaccacccaccccagcaaggacactgagcaagagaggccctatcccaactcggcccccaacactgagcatctccctcacaatttccatcccagacccccataataacaggaggggcctagggagccctccctactctcttgaataccatcaataaagttcgctgcacccacaaaaaaaaaaaaaaaaaaaaaa

SEQ ID NO: 2 - DNA, 2525 bp, Mus musculus
ctgctcactcgctcctgctcgggcttcccacagctccctcccttcctccctccctcccgaccgagggcggcccgggccggtgcatggtcagccagctcccatctctagctccccggcctctcagcagcacggaacggacggcagcggcgggcgaggaggaagatggcgggacggctgccggcctgtgtggtggactgtggcacgggatatacaaaactaggatatgctggaaatacagagccacagtttatcatcccatcatgtattgccattaaagagtctgcaaaagtgggtgaccaagcccagaggagggtgatgaaaggcgtggatgacctagacttcttcattggtgatgaagcaatagaaaagcccacatatgcaacaaagtggccaattcgccatggtatagttgaagactgggacttaatggaaaggtttatggagcaagtgatttttaaatatttaagggcagaacctgaagatcattactttcttttgactgaacctccactgaatactccagaaaacagggaatatactgctgaaataatgtttgaatccttcaatgttccaggcttgtacattgctgtgcaggctgttcttgccttagctgcatcctggacctcaagacaagtaggagagcggacgctgacgggtacagtaatagacagtggagacggagtcactcatgtcattcctgtggctgaaggatatgttatcggcagctgtattaaacacattccaatcgcaggaagagatataacatattttattcagcaactgctgcgagaccgagaagtaggaatccctcctgagcagtccttggaaactgcgaaagcagtgaaggaacgctacagttatgtctgcccagatttagtaaaagagtttaacaagtatgacaccgatgggtcaaagtggatcaaacagtacaccggagtcaacgccatctcaaagaaagagttttctattgatgttggctatgagcgattcctgggacccgagatctttttccatccagagtttgctaatccagattttacacaacctatctcagaagttgtagatgaagtcattcagaattgccccattgatgtccggcgtcctctctacaagaacattgtcctctctggtggttcaaccatgttcagggactttggacgtcgtttgcaaagagatttgaagagaactgtagatgccaggctgaagttaagcgaggagctgagtggtggtagattgaagcccaagcctattgatgtacaagttattacacaccatatgcagcggtatgcagtctggtttggagggtcaatgctggcttccacgcctgagttctaccaagtatgccacaccaaaaaggattatgaagaaattggacctagcatttgtcgtcacaatccagtgtttggagtcatgtcctaaagttgacttcttgttgattgggttagggagatggaaatgagataatctttctgatgacctgtttttgtctgaatggatggttctgaggtttttaaccaaacatgatcatacaggaatatttaatgagtgtgtcaacatgcagatgtagaagagagctaagacgattgttttcctttaggttgaatatttgaatcttatgtgtatcaaaaaagaaatgggttttagttctttctttgccctgatattttgtatattattgacttacccagtgtgctgggctcagtgggtgtgtagagagctctgtaatgcctgattgggcactgctgagtgggtagtgcctgagcttgtttatatttcatacttttttatactttgaggaaaaaaagtcaaagaaaaactgtagtattggagggaaacaatgtgaccaaggaaaagatgagttcaacaagcagcctcatgggacttggcgcacactctgggttccagttatctcgagctgctccacccctccccagcccaacggttctctctgcaaatgcttggatctaagaagctagtctcctgggttagccgatgcctgccctgctttctggttacttacattctgtttcttgctttaaaagaaggacaagactgttggaccagtattgcaattctgtagagtcgtttcttattaaaacaataatgtgattaccaaaattggcatatttaaggcctaatgccattctaataaaggcaaaaaatttctttttactacttgtttcaggctctttgatctctttataagttaactaatacgtctattttcttcagattctgcagtagctctttaaaagcacagcggttggatagcaagctgacttttttatgtgctctgtacaaataattgtgaacttttaatatgttgagtgctttcattttgataactggatctccatttgatatttcatttgtataactcatttgcagtctgaattttttttagtgccagtccctgacatatcatgaaaagttggttttctttgcattttcaaatatctggatcatgaagaaaaagtgatgacaataaattaaaattgaattacccttaaaaaaaaaaaaaaaaa

SEQ ID NO: 3 - DNA, 4872 bp, Mus musculus
gaccaaataagcagctggaggctcctgcggctgcgccccagcctgagtgagccaagacccttaatcaggcccgcccctctcagccggctctagccggctccagcggggtctctcttggagccctgccctagcttcctcgcttacaaccgaaatttagtttgcaacttttgttcttgagacttttcccccagggcttctaggaggacagcggacctctgggacatttaaggacttgggttcctcggctcccaggagctcaagtcctaagtgtcctgacagaagaccggcgggctcttagggtttgctaggaaactacgagacgatgggagaaccgctgtccactcaaaaccttcagctcggaaatgactaactgaaacgtggtttggatgaatgggtctccagtcttaaccaacgccgctcctggatgaaagttgaggtagaccttgcaggcatccaggcaaggaagggaaccagtgaagaaatcagaatgaaaacctgaagtagattgacttggatcatacttcagggaggataatcctttttcgtatttaatgctatgaatttaaagactgcctttgattggttactgtacaaaaacatcgcactactgggataggccataaaattccagaaggtcgagatttcaggttgcaacattatacctactgattctttggtggactatcaacccctgaaaggtcaatgaagtcacatctagcatcctgtctgtggccttttcagaactcaagctcttctttctgatctacttttgacatcttttcccttggacaagctgccagaagtctgatccaaattactgctgaaaataacctcaccattgtacacaggaattatactttatggctacagtgcatgttcgtagaaagaactacgtggatcaagcatgtgaggccataagcagtacttactgaggaacaccagggtcgacggatttggatgtaccaatgtcttcattctatagtgtcccatccttttaccatcccagagaaaatgttgctagaagagaatattgtgtggaatgagaccttttcccatattttatgagcaatatgcattaagaaatgcatctattctacagagctctatagccctgaagaagcaagcagccatatacccacagaccagagaacatgagactgtttgttagcagaagagtcaaaagatggaaaatttttcacttttttgtcacttgttttatattaagcttcatggttttttggagcccaatcaataattacatcatgagccatatgaagtcctactcctacagatacctcgtaaatagctatggctttgtaaacaattccctgtctctcaagcacagctctgtgcagcctcactacccatacttgatcaaccacagagagaagtgtcaggctcaagatgtcctcctcttactgtttataaagactgcccctgaaaactatggccgacgttctgcaatcagaaaaacgtggggcaatgagaactatgttcagtctcaactcaatgccaacatcaaaattctgtttgcattaggaactcctggtccactgaagggaaaagaactgcaaaaaagactaatcggggaagatcaagtgtacaaggatataattcagcaagatttcattgattctttccacaatcttacttctaaattccttcttcagttcagctgggcaaataccttttgtccacatgccaaattcctgatgactgctgatgatgatatatttatccacatgccaaatctcattgaatatcttcaagggctagagcagattggagttcgagacttttggattggtcacgttcatcgaggtggccctcctgttagggataaaagcagcaaatactatgttccctatgaaatgtacaagtggccagcctaccctgactatacagctggtgctgcctatgttgtctccagagatgtagctgccaaaatctatgaggcatcgcagacactgaattccagtatgtacatagatgatgtattcatgggcctctgtgccaataaagtgggaatcttgccacaggaccatgtatttttctctggggaagggaaaattccttatcacccctgcatctatgaaaagatgatgacatctcacggacacttacaagatctgcaggacctctggatagaggctacacatcctaaagtaaagaacatttcaaaagggttttttggtcaaatatactgcaggttaattaagatagttcttctctgcagactgacttacaggaattcatacccttgttgggctgcatttgcttagtagtacatgaatgctgtactgtgttaagtgtcactgagccaaacttgaatggaaaataataatctgtaaatgtttctctctaccctaagtcaaatgaattgaaagcaaagagattcatttcaaaacccagtcaaactggtcagtgtaactccaaattaaactgcagcttaatttcatttcaaggaattcatattctaaacgacttagatcatgagcaagggtaaagtaagaatttcaagttctcatttggttctacacatgtatttaaggtatggagaacacctgagaagccttgatatcaaaataatagtttggggaagatacctgagaaatatataatatagcatttctaaaaaattaatacctcttagaacaagcaatcccaattatcttattaaagagttctaggtagaggtggagggaggcaggtgatgcagaaggaacgtgatgaaaggttcatccgaatgaggacatatgtggaaaatgtatataatttcttatagttaagtcgtttaacattggcattcctacagccagttacctaggcagttctggaatcatttaagatatatttgtttcattttctcaaatttaatttttagtgtaaaaactttagaaagtggtcagctctaccctaataaaaactatatgtacgctttctctaaagttataaaacaagaatagttcctggcccctggaggaccttctttaaagataaatcactatcaactccaataagcttgttttaatttaatgaattattcaatggcttttaaaacactagatattggcctctgtttaaggtagttgtttggatgcagcttacaatgaggagtataataacagaagagacttaaaggaaagatagaactaggggtatagctcagcatccaaaagggaagacaaagaagcaataaaagtctcttttccttgtaattgtaacatcagcttggagtccagaccatggcactcaatactcaaatgccgcttccaataaggtcttgtaggacacagattttgttccaagtgaagacttttagcttgaaagtttttaatatagtaccacgtctaaaataatccattatttgactcaatagctatcagaggttatcttttaaatatttcaaacatgggagcaaaataagagtgctagagaaactgaccaaatgagattagaaatgttggtaaatctgcatgttctttgtaatcagaggcttggcagatctaataaattaaattacatttattttacaaaataggattaaaacatgtggctggtatgcaaacagtgcaccagcacagaaaggactaaggggtattcatcgattaaggacctgtaatgcaggtcaaaacacgatttcagcaaatgaagatggatgcttaacaaccctcagacagatgcatataatagggaaagtttagctactattattagggtggttttggaaacaatgcaaaaaaatctaaaaatacacaactaaatgatatctgtgttttcacaggtagattttttttaaaaaaatgtttacagaaatctttgtgaagttttttattatgagatatgaagcttgtttatcctactcgggtagtttcctactaatttcaagactagtgttaaacattctaaggtagttatcttagggtagattcaaggttttagatgactaacagttcagattttctgatcaattttttaaacactagagaataaaagtgtactagagaataaaagcagcttcatagttaattctcaccaattggccctttgctagctgctggctttaggtacacataggataatatgtgtccacgtttctacttggaactggtaaaagttgtcactggctggaaaatggtatctctctcttgtatacaagatggtccattgacactggtactttatgaagcagttctttgtttgtttgattgagctctcttgaaccttgttcatcttttagtttttgcttagaatagaatggaactggtttgaagttaaaggaaatattcattttgaaacttgttcattttgaaaggaaatgcaagtttcaaaatgaaaaataaaatgaaaaaggaaataaattattgtcccagatggtcacttgagttttaaaaaatggctgcacacagtaaaactgctaaaaacaaaaacttacctcattattggtttgcatcttttttcagctactaattttataccaaaatgttaaatatttatattgtttgagtttcaatcttgtatggaaaaaaataattagtaggtctaaaaatgccatgctttccaataaagaagttaaaaaaatcatcagtaatgtgaatttaaaatcctgtgtgggcatcattctgtgtatcgtcacctgctgtcttataccagaagaacctgcactacccatccattgtcctatgtgtgctcaccctgctgctgtgctcactggtgggctgtcatggagctacttaaggagctatgctgtgactgtgggaaatgctgtttgtccagaatgccatgacaaaaagctcttgcagttttagtgtattaagatgccatctaaagaggttagactctattcattagctgaataaaagaagaacttgg

SEQ ID NO: 4 - DNA, 3731 bp, Mus musculus
ttccaagcggtcaagtatgcctagagtttagcagagccagccgagaggaaatggctggttgctaaatgggaactcagagctaagtcctcttcttccttagtgtgagtgtaggggtcctgagaggttagacaaggagggaaggaagaaaagaaggaaggaaggaaggaaggaaggaaggaaggaaggaaggaaggaaggaaggaaggagagagacacctacaacccggggtttgttttcttgcggaaggaaaggggcacctacaatcccgagtttgttcccttgagggaaggacgctaaattgggcgcgcgctgaagccgccccgcaggtccgcaggtgggtagcctaggcgagggaggaaggggacattaccttcccctcggaagagggcgctggctcccccaactggcctttataaccaggccgcgggaggcgaggaggcagccggttgccgtctgcgattcgctaaagcatgcagttgggagagcagctcctggtgagctcggtgaacctgcccggcgcgcacttctactcgctggagagtgctcgcggaggaggaggaggaggaggcggaggaggagggggaggcggagggagcgtcagcctcctccccggtgctgccccctcgccccagaggctggacttagacaaagcgtccaagaagtttccgggcagtctcccgtgccaggcggggagcgcagaacccgcaggcgccggcgcgggggcccccgcggccatgctcagtgacgcggacgctggggacaccttcgggagcacctcggcggtggccaagcccgggcccccggacggccgcaagggctccccgtgcgcggaggaggagctgccctccgccgccaccgccgcggccaccgcgcgctactccatggacagcctgagctccgagcgctactacctcccgtcgccgggaccgcagggctccgagctcgccgcgccctgctcgctcttccagtacccggcggcggccggagcagcccacggacccgtgtaccccgcgtccaatggcgcgcgctacccctacggctccatgctgccccccggtggattccccgccgccgtgtgcccgcccgcgagggcgcagttcggccccgctgcgggttcggggagcggcgctggtagcagcggcggtggtgccggcggtcctggcgcctatccctacggccagggttctccgctctacgggccatacgccggaacctcagcggccgggtcttgtggaggattggggggcctaggggtgcctggctccggcttccgcgcccacgtctacctgtgcaaccggcccctatggctcaaattccaccggcaccaaactgagatgatcatcaccaaacagggcaggcgcatgtttcctttcttgagcttcaacataaacggactcaaccccaccgcccactacaatgttttcgtggaagtggttctggccgaccctaaccactggcgcttccaggggggcaagtgggtgacctgcggcaaagcggacaataacatgcagggcaataagatgtacgttcacccagaatctcctaacactggctcccactggatgaggcaggagatttcctttgggaagttaaaactcaccaataacaaaggtgcaaacaacaacaacacacagatgatagtgttgcagtctctgcacaaataccaaccgaggctgcacatcgtggaagtgacagaggacggtgtggaggacttgaatgaaccttccaagactcagaccttcaccttctcagagacacagttcatcgctgtgacggcctaccaaaacacggatatcacccagctaaagatcgaccataaccccttcgccaaaggcttccgggacaactacgattccatgtacacggcttcagaaaatgacaggttaactccatctcccacggattcccctagatcccatcagattgtccctggaggtcggtacggcgttcaaaacttcttcccggagccctttgtcaacactttgcctcaagcccgatattataacggtgagagaaccgtgccacagaccaacggcctcctctcaccccaacagagcgaagaggtggccaaccctccccagcggtggcttgtcacgcctgtccagcaacctgtgaccaacaagctagacatcggttcttatgaatctgaatatacttccagtaccttgctcccatatggtattaagtccttgcccctgcagacatcccatgccctggggtattaccctgacccgaccttccctgctatggcagggtggggaggccgtggcgcttatcagaggaagatggcagctggactaccatggacatccagaatgagcccacctgtcttcccagaagatcagcttgccaaggaaaaagttaaagaagagattagttcctcctggatagagactcccccctccatcaagtctctagactccagcgactccggggtgtacaacagcgcttgcaagagaaagcgcctgtctcccagcacccccagcaatggaaactcgccccccataaagtgtgaggacattaacactgaagagtacagtaaagacacctccaaaggcatgggggcttattatgctttttacacaagtccctagagcattattttaacctttaacctttccagatggacctggtggtgttttgttgtgttctttgcctaggttaccaaaaagacttttgccttccacctttgatgtatcctgttttgtgcaattctctgagagaaggcgccaaagctttgccacggccgcaggtaactaaactgaagcagacctagcattggttttaaagtgactggaggacgcaggagtgtttttaaaactgaaaaggtcgttcaaggtgctggattgattcatttatgggaaacgagaaatgttcagaaaaagcaggctatgaagaacgagtgcccggtgctattaaatgaatttcaaagacaagaaaacatgacagaaaaaaacaaaacaaaacaaactttggctcttatttctactcataaatctttaagcaaacagcagcccagagggttaaaatatttttgcttcgggaagagaagggttggaccttgagttaacagtgggctcttcacagatatcttctgccagggcctatagaggctgagcctgagagtcaagcggggaaaacaaacaaacaaacaaactgatgtttaagtggctagctagtactcactgccttcaattccttgcagaattttggaataaaccatttgttgtagggctggctctgtgtggctgggatgacctgtagaaggcaggaggatcagaaagaaagggagaagttaagtgtacaattgtatgtttagacaccttagaatattctggaatatattgtacaaattcaactgcctaggtatcagataaaaataatgtaaatttggtgctttggcgttgtaaagaatttgcaaaaccacaactgcagttaagaggagggattcatatcttggagatattctgtccacttcgctaagtactcagctgcctccgtacttgcttctacacacttaaggaaaaaccctgcttaatcctctttccttggtcatccccacttcccccttcagtaaatgtgaaaaaaaaaattgcagtatgatgtcacagttcctataggctttttagagattctgaatttttatgtacagtcataagtcatttttaataaatgtggttcaataagggaccaggtatagctctgtcttctgtaaaaaaaaaaaaaaaaa

SEQ ID NO: 5 - DNA, 2145 bp, Mus musculus
tccgcgagccaacctgcggcgggtcatccccgcctctacagcttactggcaaggaggtgggaggaaagaaggaagagaggggaggaggcaggacggagggagggactgcccgggaggcagaagctctgcaaggagccgacggagcaccgtgggctgaggtgcagccagctacctttatctctagccccctgcgcctcgcgcctctggcagccttcaacgtttgtccccagacagcatggtgaggtctgctctgggtccctcgccaccatgtacgtgagctaccttctggacaaggacgtgagcatgtatcctagctccgtgcgccactccggcggcctgaacctggctccgcagaactttgtcagtcctccgcagtacccggactacggtggttaccacgtggcggccgcggcggctgctacggcgaacttggacagcgctcagtccccagggccatcctggcccaccgcgtacggcgcccctctccgcgaggactggaatggctacgcacccgggggcgctgcggcagccaacgcggtagcccacggtctcaatggtggctccccggccgccgctatgggctacagcagccccgccgaataccacgcgcaccatcacccgcatcatcacccgcaccatccggccgcctcgccgtcctgcgcctccggcttgctgcagacgctcaacctcggccccccggggcccgcagccaccgccgccgccgaacagctgtcccccagcggccagcggcgaaacctgtgcgagtggatgcggaagcccgcgcagcagtccctaggaagccaagtgaaaaccaggacaaaagacaaataccgggtggtgtacacagaccatcagcggctggagctggagaaggagtttcactttagtcgatacatcaccatcaggaggaaaagtgagctggctgccacacttgggctctccgagaggcaggttaaaatttggtttcagaaccgcagagccaaggagaggaaaatcaagaagaagcagcagcagcaacagcagcagcagcaacaacagcctccacagccgccgccacaaccttcccagcctcagccgggtgccctgcggagcgtgcccgagcccttgagtcctgtgacctccttgcaaggctcagtgcctggttctgtccctggggttctggggccagctggaggggttttaaactccactgtcacccagtgacccctcccgtggtctgaagcggcggcggcacagcaatcccaggctgagccatgaggagtatggacgctgcgagaatcctcagaagagattcctctcctcctacccacgaacagcatctactgatggagattgaggacagaagatgagtggaattatggacctcaggggaagacatggtttagattttttttttctttttaacttttcccattccgactcttcctgccagcaacgacaaacgaagtgattcctggggcttcttcgttcatgctctttgccaggactgactaccgacatgaagctatcagcctcttttgccccagctctttgcctctctgtatttctgtgtggagctgaggagagagtgagactggatggggtgggggtagcaatacttgagccaaggtggctgtttcctgctgactgctttctgagaaccagctggccgtcctgcctccgggccagggactattcaaactacaggagccagaggcagctaagatagctggactgaccgaagtctgcagaacctcccccaccaggtggtctgggctttcttctccacaaatcaggaaggggtggtgggttcaggggctgcggtgagagggggttggttagccaacgccaggcccctgcgacaagggcttgtttagaaagcctgtcaccagagctgctgtaggcggaatgtatgtctgtgttgtaaatgccagagccaacctggacttcctgtcccttccctcgtctttggctgaagaagaccggaattgtttgctgctgttcgagtcactgatctgtgtaacgagccaaacaagccttttaaaaagccttcttgatccatgggtagagaagttgtatggtgaagggaagtcgggagggggggaaggggatccgaacacagttgacttttattttgtaaaaagacaaagataaacgaactttaac

SEQ ID NO: 6 - DNA, 3513 bp, Mus musculus
agacgcacttctcagtacgcggagagcggctcggccgcgcgcacgccgggtatccaggccgagccgggaacgccgagagcatggcggtcgcgggcgccaagaggcgcgcggttgcgaccccggcggcggcggcggcggaggaggagcggcaggcgcgggagaagatgttggaggcgcggcgcggggacggcgcggaccccgagggcgaaggcgtgaccctgcagcgcaatatcacgctgctcaacggtgtggccatcatcgtgggcaccatcatcggctctggcatcttcgtgacgcccaccggcgtgctcaaggaagcggggtcgcccggactgtcgctcgtggtgtgggctgtgtgcggcgtcttctccatcgtgggcgcactctgctacgcggagctgggcaccaccatctccaagtcaggcggcgactacgcctacatgctggaggtctacggctcgttgcccgccttcctcaagctctggatcgagctgctcatcattcggccctcctcgcagtacatcgtggcgctggtcttcgccacctacttgctcaagccggtcttccccacttgtccggtgcccgaggaagctgccaagctcgtggcctgcctctgcgtgctgctcctcacagctgtgaactgctacagcgtaaaggctgcgacccgtgtgcaggacgcctttgcggctgccaagctgctggccctggccctcatcattttgctcggcttcatccagatggggaaggacatgggacaaggtgatgcgtccaacctgcagcagaagttgtcctttgaaggcaccaatctggacgtggggaacattgtgttggctttgtacagcggcctcttcgcctacggaggatggaactatctgaattttgtcacggaggagatgatcaacccctacaggaacctgcccctggccatcatcatctccttgcccattgtcacactggtctatgtgctgacgaacctggcctatttcactaccctctctaccaaccagatgctgacatctgaagctgtggctgtggacttcgggaactaccacttgggtgtcatgtcctggatcattcctgtctttgtgggcttgtcttgcttcggctctgtcaatgggtctctgttcacgtcctcaagactgttcttcgtgggatccagggagggccacctgccttctgtcctctccatgatccacccacagctgctgacacctgtgccatcactggtgttcacgtgtatcatgacgctgatgtacgccttctccagagatatcttctccatcatcaacttcttcagcttcttcaactggctgtgtgtggccctggccatcatcggtatgatgtggctccgattcaagaagcctgagctggagcgtcccatcaaggtgaatctggccctcccggtgttctttatcctggcctgcctcttcctcattgccgtgtcattctggaagacacccatggagtgtggcattggcttcgccatcatcctcagcgggctgcctgtctacttctttggtgtctggtggaagaacaagcccaagtggattctgcaggctatcttctctgtgacggtgctctgccagaagttgatgcaggtggtacctcaggagacttagtcacgtgtcctgggtgctgccgcgctgtgcctgctccagacaactctcctttggaagatcagcggccaggcctgtcagccccacagctccagtgagcaccaccagccgcctaacacaatccactgtccctcaaacgtcaggtgtccacagtggctgtgaaagaaaactggtacgatttggtcccagaaggtgaccgtccatggtccatgcagacacagcacccagtatgaggcgtgctgggacctgaggcctggactttctgactttctggggactgccacatccaggctttctcccctgtgatttttgttttgttttggtttttgtagcgtgcatttgggtcaagtttacactagcgagatgattattttttaacaaaacagggtagcaaggagcaggagatgccgtggccgcacagtccggccctgtgggaactgctggccacagctcttttcccaaggctgtcgttcagtagcatagagtgcctcaaggggaccgcatcactgtcaccaggtcagaagaactactgagacttgcacatcacctgttccactgtggacttggactgacagatggacgacacatatgctagcttggactttagttttctgggaccctagagatggaaccccaacctttctcatgagacagagctctactgtgtgggtcacttgttaggttgaaaatgtagagctcagctctcgccgccctgaagtcctggctccatatgaaagatctctgcagccatgaccctaacagcacctagctgtccgctgcagggccttgtgctctgtgcacacaatgcagcagtgccccggctctcacacgcacagcactccccgccctgccccacctctcttagaaccagaccagcaccatggctctcccacccccacccccacccccaagcacctggtctgtccccattgttcttgagactgctagtagcattctgtcaaagtcactactgctccctccagccccacccttagccattgttcagaaaaataggcaccaagcacccctctgctctctgccaccagtgtggtgtgaccactccagctccctgagtatgaaagccactggctacatgtgtgggaccatcctgcagccttcccgttgtgctctgggtgagacagttgggtccagccagcacatcactgaaggagctgatctgctggagggacttcaggatgtgcgtcccctgctggcctctgccattgcctcagaccatggcctccctgaaggggtgtctactggaatggctactatgcagttcttacatagtggtgacttaccactggccaccctgacctctcctctgtgcttctggggttccctttcattggggtgaggaagcaaggtcaaggtcagagtgcctcttgaaggttctcccagatatgggaactggggtctcttccctggaaatgagggcagggacccagttccatcctggggtgtgtccccagatacagcctgggttaaagggcagggattcatggtgctgacccaggctctctgagaaggccctgtcattcctgtttgtgatgttctgaggatgcccatctgtaggtttttattttcaaatcaagctccgaccaccgccattttgcccagtgctttctattttggttttatcctaacatgtcactgccctgaaagacacccctacctggcccctttcaatggtcagtgtctgacccaccacactgggtccatctgctgtgccacctgagatagtgctgtggttataatgtcttttactattgtattaatggctagcctgttacattagactggggggggggtacagaggtctgctgttttgtgaggctttttgtttttttggggggttggttttgttttttttaaaagctattggaattctaagctg

SEQ ID NO: 7 - DNA, 2791 bp, Mus musculus
agagggcccagcgccactgcagccgcgccacctcccagggccgggccagcccaggcgtccgcgctctcggggtggactccccccgctgcgcgctcaagccggcgatggctcctctcggatacctcttagtgctctgcagcctgaagcaggctctgggcagctacccgatctggtggtccttggctgtgggaccccagtactcctctctgagcactcagcccattctctgtgccagcatcccaggcctggtaccgaagcagctgcgcttctgcaggaactacgtggagatcatgcccagcgtggctgagggtgtcaaagcgggcatccaggagtgccagcaccagttccgaggccggcgttggaactgcaccaccgtcagcaacagcctggccatctttggccctgttctggacaaagccacccgggagtcagcctttgtccatgccatcgcctccgctggagtagctttcgcagtgacacgctcctgtgcagagggatcagctgctatctgtgggtgcagcagccgcctccagggctccccaggcgagggctggaagtggggcggctgtagtgaggacattgaatttggaggaatggtctctcgggagtttgccgatgccagggagaaccggccggatgcccgctctgccatgaaccgtcacaacaatgaggctgggcgccaggccatcgccagtcacatgcacctcaagtgcaaatgccacgggctatctggcagctgtgaagtgaagacctgctggtggtcgcagccggacttccgcaccatcggggatttcctcaaggacaagtatgacagtgcctcggagatggtggtagagaaacaccgagagtctcgtggctgggtggagaccctgaggccacgttacacgtacttcaaggtgccgacagaacgcgacctggtctactacgaggcctcacccaacttctgcgaacctaaccccgaaaccggctccttcgggacgcgtgaccgcacctgcaatgtgagctcgcatggcatagatgggtgcgacctgttgtgctgcgggcgcgggcataacgcgcgcactgagcgacggagggagaaatgccactgtgttttccattggtgctgctacgtcagctgccaggagtgcacacgtgtctatgacgtgcacacctgcaagtaggagagctcctaacacgggagcagggttcattccgaggggcaaggttcctacctgggggcggggttcctacttggaggggtctcttacttggggactcggttcttacttgagggcggagatcctacctgtgagggtctcatacctaaggacccggtttctgccttcagcctgggctcctatttgggatctgggttcctttttaggggagaagctcctgtctgggatacgggtttctgcccgagggtggggctccacttggggatggaattccaatttgggccggaagtcctacctcaatggcttggactcctctcttgacccgacagggctcaaatggagacaggtaagctactccctcaactaggtggggttcgtgcggatgggtgggaggggagagattagggtccctcctcccagaggcactgctctatctagatacatgagagggtgcttcagggtgggccctatttgggcttgaggatcccgtgggggcggggcttcaccccgactgggtggaacttttggagacccccttccactggggcaaggcttcactgaagactcatgggatggagctccacggaaggaggagttcctgagcgagcctgggctctgagcaggccatccagctcccatctggcccctttccagtcctggtgtaaggttcaacctgcaagcctcatctgcgcagagcaggatctcctggcagaatgaggcatggagaagaactcaggggtgataccaagacctaacaaaccccgtgcctgggtacctcttttaaagctctgcaccccttcttcaagggctttcctagtctccttggcagagctttcctgaggaagatttgcagtcccccagagttcaagtgaacacccatagaacagaacagactctatcctgagtagagagggttctctaggaatctctatggggactgctaggaaggatcctgggcatgacagcctcgtatgatagcctgcatccgctctgacacttaatactcagatctcccgggaaacccagctcatccggtccgtgatgtccatgccccaaatgcctcagagatgttgcctcactttgagttgtatgaacttcggagacatggggacacagtcaagccgcagagccagggttgtttcaggacccatctgattccccagagcctgctgttgaggcaatggtcaccagatccgttggccaccaccctgtcccgagcttctctagtgtctgtctggcctggaagtgaggtgctacatacagcccatctgccacaagagcttcctgattggtaccactgtgaaccgtccctcccccctccagacaggggaggggatgtggccatacaggagtgtgcctggagagcgcggaaagaggaagagaggctgcacacgcgtggtgactgactgtcttctgcctggaactttgcgttcgcgcttgtaactttattttcaatgctgctatatccacccaccactggatttagacaaaagtgattttctttttttttttttcttttctttctatgaaagaaattattttagtttatagtatgtttgtttcaaataatggggaaagtaaaaagagagaaaaaaaaaaaaaaaaaaaaaaaaaaaaaaaaaaaaaaaaaa

SEQ ID NO: 8 - DNA, 4273 bp, Mus musculus
agtccctggaagcagacgtttcggccacagacccagagaggaggagctgacaatcaggaggcgtgagccgcctggagtctgcagaattcgtggtgtgaatgaactgggggcatcttgggcacagggattgccccccctccttccccgcctcgggccacagttgagtagtggggcattttttttcaccttcttgtgaagaattttttttattatttgttgtaaagtcttttgcacaatcacgcccacatttggggttggaaagccctaattaccgccgtcgctgatggacgttagagagggagcgcctcgccgcggaacagtcgcctgcgcgccctcgtcggacccgcggctcctgcactgtgtccccgctcggccctgcgcttgctgctcgcccgcgcgcgccggcgccctctcggttcctgggcacatttccacgctataccaactcctctgcccgagtccgggcgccagtgctcgcttccgctccgggtcgctgcgcccacccgacgcgcccaggaggactccgcagccctgctttggattgtcccccaaggcttaaccccgacgcttcgcttgaattcctcggccgccttcgctcgggtggcgacttcctctccgtgccccctccccctcgccatgaagaagcccattggaatattaagcccgggagtggctttggggaccgctggaggtgccatgtcttccaagttcttcctaatggctttggccacgtttttctccttcgcccaggttgttatagaagctaattcttggtggtctctaggtatgaataaccctgttcagatgtcagaagtatatatcataggtgcacagcctctctgcagccaactggcaggactttctcaaggacagaagaaactctgccacttgtatcaggaccacatgcagtacattggagaaggtgcgaagacaggcatcaaggaatgccagtaccagttccggcatcggagatggaactgcagcacagtggacaatacttctgtctttggcagggtgatgcaaataggcagccgagagacggccttcacgtacgcggtgagcgcagctggggtggtgaacgccatgagccgagcatgccgggagggcgagctgtctacctgtggctgcagccgcgctgcgcgccccaaggacctgcctcgggactggttgtggggcggctgcggagacaacatcgactatggctaccgcttcgccaaggagttcgtggacgctagagaaagggaacgaatccacgctaagggttcctatgagagcgcacgcatcctcatgaacttacacaacaatgaagcaggccgtaggacagtatacaacctggcagatgtagcctgtaagtgtcatggagtgtctggctcctgtagcctcaagacgtgctggctgcagctggcggacttccggaaggtgggcgatgccctcaaggagaagtatgatagcgcggcggccatgaggctcaacagccggggcaagctggtgcaggtcaacagccgcttcaactccccgaccacgcaggacctggtctacatcgaccccagtccggactactgtgtgcgcaacgagagcactggctcgctgggcacgcagggacgcctgtgcaacaagacctcagaggggatggacggctgcgagctcatgtgctgtgggcgtggctatgaccagtttaagacagtgcagaccgaacgctgtcattgcaagtttcactggtgctgctatgtcaaatgcaagaagtgcacggagattgtggatcagttcgtgtgcaaatagtggtgtgcctgcccttcacccagtcccactcccaggacccacttatttatagaaagtacagtgcttctggttctttttatttctcccccaagaattgcagctggaaccatgtgttttgttttgttttattttgttttttcttttctgttaccatctaagaactctgtggtttattattaatattataattaatatttggcaatagtgggggaaactaagaaaaatatttattttgaggatctttgcaaagttagtacaaaatttctttcttctgatgctacaggataaaggggaaaaactatgtattcgaacttagctgtgcagttgggggttcacatctagaaggtgtaggagccattttcttctcaaacagagagtcctttgagatgggtggtatccaggtgaaggaggaggtacagacccatgaataacagttcctgtgaccaaaatgaattgcaggtgctctggtacaaaagatcttaaatatagatatattaaatatacatatatgccaaaaatacagaatatgagacactccctaacccagaggttaccagcctggttttgtgggttttttgttttgttttgttttttctttttttgggttttgtttgtttgtttgtttgtttgtatttttggtgtgtgtgtgtgtgtatttctagaatgatcttttagaaggtacaagcaagaatctcatatcttcagaagcaggcatatcatgtatgttactgtgtcccacctacagatactccattcatgaatgggcctttttctaacagttcatgaatattggggagccggtgggctgggggagggaggtccccagaaattagaaaacttgaagtttcctacattgaggccataatcttgtgttagcccagctgattcttaataccagacttttagatccataaaggaatttttgactaaaaaaaaaaaatcttgttttgaaagccatcttattttcttaaaaatgaaaaattacccatgaatcccatttgcaacccctcacccccacaggcaacaagaaagtcccatgtagttgagcactgcgaacacctctgtgaggagatgatggcagccatcttcctgcatgatcccatgccctttctggactctctgctggccatgcttccgaatggcagccctggtggacactcactgctggtagggcagaaaatgtacacgaggagccatgttcagaaccagccacttaggggttgttctctgaggcttttctttggaggtacggtaacttgatgtgttttgatgatatctcttggcccagggagtccacagaggtgttgcagctgtttggttgttatcctcctgcgtttagactttccatttgtgcttttcctattaccctgcaggtgtaccctaaaactgttcctagtgtacttgaacagttgcatttataaggggggatgtggtttaatggtgcctgatatctcagtttttttgtatataacatatatataaatatacatatataaatatagatataattatatctcagtgcagtctgggatttagacctacagttttctctgggcttgctctctgcctggagtatcgtccttcattgcagtccaattgggatttctttttttccaaaaattttgagtcttaacattgacctgtgacaggatcctaccacgaataccaggaagcaagctaagactcggaggaagctctcagggctcatgtcctgaatgtatgttggttagaaagtagcctttctgcttcctgcccatggccagttctccaccctctctttggtgttctttgtggggagggcactgtggtttgtcgcagccctggacttcgagaggctcccagaacccaggatcaccagcctcctgtctgtttgcttcactcctttcccagggaggacttgggactgtcctgtctgacaggacggatctgagttcccgaagcaaaccagctcaccacatagatagctagtttaaacaatgttttaaaataagggcacctctgtttcaaaagtgacatctgctgtgttgttttcgaggcctgatactcttacaaggtttgaaaaaaaatgtgtgtatccattcatgggcttggtagccttctggtcacctcagtcctgtggctcttaacttattgcccaacaatattcatttcccctcagctacaatgaattgcaagcaaaagatgttgaaaaaaagcactaatttagtttaaaatgtcactttttggtttttattctacaaaaaccatgaagttctctctctctctctctctctctcttatttgttaaatcagattatgttctttttttgtttttgtttttagtgattcatgtttatgagcagagtggagtttaacaatcctagctttaaaaaaaacctatttaatgtaagatattctacgcatccttcagatattttgtatatcccctatggcctttattctgtacttttaatgtacatatttctgtcttgtgtgatttgtatatttcactggt

SEQ ID NO: 9 - DNA, 3691 bp, Mus musculus
gcaaactggacattagcttctcctgtgagaaccttccagccttttcctgtcttcatcctcttccagccccagcggcctccttacccaacttcaggtaccaatggggatcccagtggggaagtcgatgttggtgcttctcatctctttggccttcgccttgtgctgcatcgctgcttacggccccggagagactctgtgcggaggggagcttgttgacacgcttcagtttgtctgttcggaccgcggcttctacttcagcaggccttcaagccgtgccaaccgtcgcagccgtggcatcgtggaagagtgctgcttccgcagctgcgacctggccctcctggagacatactgtgccacccccgccaagtccgagagggacgtgtctacctctcaggccgtacttccggacgacttccccagataccccgtgggcaagttcttccaatatgacacctggagacagtccgcgggacgcctgcgcagaggcctgcctgccctcctgcgtgcccgccggggtcgcatgcttgccaaagagctcaaagagttcagagaggccaaacgtcatcgtcccctgatcgtgttaccacccaaagaccccgcccacgggggagcctcttcggagatgtccagcaaccatcagtgaatcaaattatgtggtaattctgcaatgtagtaccatcaatctgtgacctcctcttgagcagggacagttccatcacgtcccacactaagatctctctgctccactccctccccaggtttctccttacccacccccatgccccgcctccccacatcaggctgttccccttgccccacaccatcgggcaaggggatctcagcagttctaaaaaaccaaatttgattggctctaaacaacccaattgacaccccccaaattatatattcacattaaaaaaaactttaaaaactacagtccctttaaaaacaaattggcttttaagaaattccataactgataatctaaaaattaaataactaaagaaattaattggctaaaaacatactaaaaattaattggcttaaaaacaattggcaaaatcaaataattcggcgccccccccccttcatcttctttccatttggatctttagtcaaattggctaagacttggatcccagaacccaagaagaaaggaaggggacccaaaattttgcaggtggcatgtcattgcttcagtgccctctccttatcccaagcacctttagtgtaaagctggctgagaacaacaatagccgcccaaactctttcttcactggtcattccatcacaaatgtcccccatgtcaccaagggggctgggtgaaagaacccaaggagaagaacagaacatgaaaactggaaatagaacttaattggcacaagcccccagtcccaaaatctcacttttccctacctaccctaaaaagcacatgattatactcacacccacaggcattaacacacacatacacataacacatatgcacacacacacacacacacacacacacacacacacagaggtatgcacacccacataattggatgaaaacattgaaatggctgaacaacttcgatttgaaccacattccccaaaacaaggcccatcccaaatttcctgagcagtttgcatggtttgagctctcctcccaatccatctagtttctgctgctgcttattggctctatgtgtgaccaggctgctagttctgtctttctcagtgagggaggtgttctagcaaggctcaattccatctaagcacctgtccacatggtgagcccggtgctccccaaccacccccgccatgaggaccaatgactagccagtgtagaataagtttggccagataaggagatggcactgccaagtgatacatgctgcccaagtaacctgacccctagttgtgctcctgggaggaaagatctgggggacaacccctaccctgagcacacctatgggccatctttgtcagtctcctggggagcccccgctttttaggggctccccaggagactcacactgatgtggggagtgtgggaagtctggcggttggaggggtgggtggggggcagtgggggctgggtggggggagctatgggtaggaagtggtcccagagaggttttaggtggaagaatcaggaggagtcacaggtcaacttgcagaattactgaagaattaggaccccaaattttatgccaattgatctattcccctctttttatttctggggccggttttttccttttttttttaatccctccttagctttttatgcgctcataatcaattgtacccattccctacataacgggagcagtgatcaggtaatgaatgcatcgagccatcaacaccagctagagccatcaacaccggctaccacaatgtcctgctctccacaaccttgatttttttttttatctctctctatcgcttggcctgagttgggagtggagtctctgtggggtgctggccacgcacccacagagaaataaaaggaattgagaaggtcgctacctggcctgacttctggggacagtggctggtccccagaagttctgaggagtggagggggcgtggggcagtgtcccctcaggtgttaggaaggtgctcggaggccacaaaagatggggctcccagctggccactgccagttgggggggaaggggatagagatgtgagagtagacaggttacatcaggcgggagcaagtggctgccttctgagcatttgggggagaacctctcccgtccctcagtgtcatcttgcccactcctcagcaccccaccttgcccccaggaggtctggagctctacagacctcctgggggcaaggtggggtgaggcccggagttggggaagctaggaggcttaaagcctacagagccaggagaactgtatacacggggtcatctgggccctggggcctgagggtctggcgagccgtagcagccactccacggtgcctaggactgcggcggggaacagggcggctggaggtttacctcacccccacttctgcttccagtgcagtccccctgcccaacagtccaactagcaatctagaggcctgaggcttctgggcccaggtgacaggactggcaccaccctgggggcggtgtgtgtcagccaagcatggcacagagggtccctcagcaagtgcctaaagaatgggccatttggaacattggacagaaactcaaagagtaaattgttataattggagactatgaattggcctggtatccaaaacatctcgaggcaccctaaattacctgcccatttggctggacacccacccagtgttaatatgcctcgtgggatgggtgctttcaggggcatttgctgaccaccctctgtgtccccacatttgcagttctccccatcataggtcaccttgatgcaggcacctccctggcctcctatgcctagtgtggccctccatcttgtctcttccctactgtcttcagtgggatcccctcttgggtccctccctttgtcatcatgtgaagacttcccacgcgtcgaacgccatatgtcacctgtgccactgtccatgtcatccagcagtggccccaggtgtttgcctcaactcagtccctctaacatgcattttctggcaaaatccaaagcttgggttttgtttttaacctgttaacgctcgcaaacctaataaagcatttaaaatacta

SEQ ID NO: 10 - DNA, 1612 bp, Mus musculus
gtgctaccctagggggtcgcgctcttctgcctcctacctcttggtcaccgcaaagcttggtccggttcttcatccggctgcaagcgctaggtgtgcggagacctggcagctcttggggcttaagggctgagcaccaggacgggtggaggtgcctgtagagtacattcggaccctctctcggaccctctctcagcccctgagtgtgcgggacctgcggagcgcagttcgggatctgcactcgaggatttttcgaggacgcaataagctaagcatctgcccggagcatggaagcacaccttgactggtacggggtcccaggactgcaggaggccagcgacgcgtgccctagggagtcctgcagcagtgccctgcctgaggcccgtgaaggtgcaaacgtccacttcccaccgcacccggttcctcgcgagcacttttcctgtgccgcaccagaactcgtagcaggggcccaggggctgaatgcaagcttgatggacggcggcgcgctgcccagactcatgcccacctcgtctggagtcgctggagcctgcgctgctcggcggagacaagcgtctccggaattgctgcgctgcagccggcggcggcgatctggagcaaccgaggccagcagcagctcggctgccgtggcacgccgcaatgagcgcgagcgcaaccgcgtaaagctggtaaacttgggcttccaggcgctgcggcagcacgtgccgcacggcggcgccaacaagaagctgagtaaggtggagacgctgcgctccgcggtagagtacattcgtgcgctgcagcggctgctcgcagagcacgacgcggtgcgtgccgcgctcgctggggggctgttaacacccgctactccgccgtccgatgagtgcgcgcagccctctgcctcccctgccagcgcgtctctgtcctgcgcctctacgtctccgtccccggaccgcctgggctgctctgagcctacctccccgcgctccgcctactcgtcggaggaaagcagctgcgagggagagctaagcccgatggagcaggagctgcttgacttttccagttggttagggggctactgagcatcccacccccctaagctgcatccctgggtgactcctggtggacctacctgcttctagcccaagaaacctgggcctatgccttacccatgctgtctagtgcagcctgaccaaatgccaagtgctgactgacctctgctcggcctccacgccgcggaatgacatcttccatcttccggaccttgcagcatcaggacttggaaatttctcaggataaagatttttacaatgacaatctactttttatcaattaacttgaactgttgtaggactctactgaaaatatgaagaattatttttatacaaaggatccttaagcttggagcacaataaagatgacctctgtccctcacccccactgtctagaactttccaacctggccaaagtgtggaccggttgggccctgagggcaagatgcctggctgcacccttcttcctcttctgaagcctatactgacgctgatgtttggccagtgtgggaaccctgctgttgcaaagtgtactattctataaaagttgtttttcattggtgttt

SEQ ID NO: 11 - DNA, 2498 bp, Mus musculus
atgggctcgggccgtcgcctccgcctggccgctctccagcccagactgtcacgggccacacggggtccacatcggcggcggcagcagcagcagcgtcctggggacggcccgaggccgcgtcccccggctgctgtcaggagctggcggcggaaatgaggcgctggccgttttccgagccggggtttcctgcctgagcgccgagaggccgtgcgacgccatgggccggccgggccccgacccgcagcttgcgcgcccggccgccccgcctcgctctccaccatgaacgagcccaccgtgcagccgtcccgcacatcctccgcacccgcctcgccggcatccccacgcggctggagcgacttctgcgagcagcacgcagcagcggcggcccgggagctggcccgccagtactggttgtttgcgcgcgcgcacccacagccgccgcgcgcggacctggtgtcgctgcagttcgcggagctcttccagcgccacttctgccgggaggtgcgcgagagcctcgcaggaccgccgggtcacgactaccgcgccactgctccgccccgccccgcgctgcccaaggcacgcagctccgaggacctgggcccgcggcccgcctgtgccctgcagcacctgcgccgcggcctgcgccagctcttccgccgccgctcggcaggggagctgcccggggctaccagtgacaccaatgacatcgacaccaccgcagccagcaggccgggcccggcccgcaagttgctaccctggggcctgcgagagccgcccactgaggcgctcaaggaggtcgtattgcgctatagcctggcggacgaggcagcaatggacagcggcgcacgctggcagcggggtcgcctggtgcttcggtctccaggtccgggccacagccactttctgcagctcttcgatccgcccaagagctcaaagcccaagctccaagaggcctgttccagcatccgggaggtccgaccatgtacacgcctggagatgcctgacaacctctacacctttgtgttgaaggtgcaggaccagacagacatcatctttgaggtgggagatgaacagcagctgaactcatggctggcagagctcagggcaagcacaggccttgggctggagcacccggacaccgagttacctctttccttagcggcagagcctggcccagctagatccccaaggggaagcactgactccctggaccaaggtgcttcacctggggtgttgctggacccagcctgccagaaaacagatcacttcctatcctgctacccctggttccacggccccatctccagggtgagggctgcacagctggtccagctccagggccctgatgcccacggcgtgttcctggtgcggcagagtgagtcccggagaggagagtatgtactcacattcaacttacagggcagagccaagcacttacgcctggtgctcacagagcgtggacagtgccgggtgcaacacctgcacttcccctcggtggtagatatgctccgccacttccagcgttctcctatcccactggaatgtggagcagcttgtgacgtccgactctctggctatgtggtagtcctctctcaggcaccaggttcctccaacaccgtcctcttccctttttcccttcctcactgggattcggagctgggtcatccccacctcagctctgttggctgtccccccagccatggtgcagaggctctccctggccaagtgacaccacctgagcagatcttccacctggtgccttctcctgaggaactggccaacagtctgcggcagctggagctcgagtctgtgagcagtgcccgggactcggactatgacatggactcctcttcacggggccaccttcgggccattgacaaccagtacacccctctctcacagctgtgcagagaggcagacgtgtgaatggaaccattttcctcccctccagagaactataggctgctgtcagccttccccagctcagatgtgccccgcttccaacacacacaaggctgtcacaggcttcctgtcagccagagctagcccccggctttctcccactgtttacagatgtagttcttgtgcacaggtgccactagctggtaccctaggcctagtccccaacccaaaaggaggtggggctacaggcccagagctggcagtggaaactgacagagctgatgacagacttcttaccaggcagggtcatcgggaagcacaaaacactaacagtccctggattctcacatggtgtccttccctgtagcttcagatctgtggcaagtcctcatgggttccatgccacaggcgatggatgcttcatgatcctgctcctactcttagctttacgacagaagctctgccagagggacaagcttaaagatcaaaattgatttaaacattttacctcagagtacttttttttgttttgttttaaaggattcaggtttccatttaatattaaagcttattcactgcaaaaaaaaaaaaaaaaa

SEQ ID NO: 12 - DNA, 1310 bp, Homo sapiens
aaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 13 - DNA, 1635 bp, Homo sapiens
aactctatatagggagttcaactggtcacccagagctgtcctgtggcctctgcagctcagcatggctagggtactgggagcacccgttgcactggggttgtggagcctatgctggtctctggccattgccacccctcttcctccgactagtgcccatgggaatgttgctgaaggcgagaccaagccagacccagacgtgactgaacgctgctcagatggctggagctttgatgctaccaccctggatgacaatggaaccatgctgttttttaaaggggagtttgtgtggaagagtcacaaatgggaccgggagttaatctcagagagatggaagaatttccccagccctgtggatgctgcattccgtcaaggtcacaacagtgtctttctgatcaagggggacaaagtctgggtataccctcctgaaaagaaggagaaaggatacccaaagttgctccaagatgaatttcctggaatcccatccccactggatgcagctgtggaatgtcaccgtggagaatgtcaagctgaaggcgtcctcttcttccaaggtgaccgcgagtggttctgggacttggctacgggaaccatgaaggagcgttcctggccagctgttgggaactgctcctctgccctgagatggctgggccgctactactgcttccagggtaaccaattcctgcgcttcgaccctgtcaggggagaggtgcctcccaggtacccgcgggatgtccgagactacttcatgccctgccctggcagaggccatggacacaggaatgggactggccatgggaacagtacccaccatggccctgagtatatgcgctgtagcccacatctagtcttgtctgcactgacgtctgacaaccatggtgccacctatgccttcagtgggacccactactggcgtctggacaccagccgggatggctggcatagctggcccattgctcatcagtggccccagggtccttcagcagtggatgctgccttttcctgggaagaaaaactctatctggtccagggcacccaggtatatgtcttcctgacaaagggaggctataccctagtaagcggttatccgaagcggctggagaaggaagtcgggacccctcatgggattatcctggactctgtggatgcggcctttatctgccctgggtcttctcggctccatatcatggcaggacggcggctgtggtggctggacctgaagtcaggagcccaagccacgtggacagagcttccttggccccatgagaaggtagacggagccttgtgtatggaaaagtcccttggccctaactcatgttccgccaatggtcccggcttgtacctcatccatggtcccaatttgtactgctacagtgatgtggagaaactgaatgcagccaaggcccttccgcaaccccagaatgtgaccagtctcctgggctgcactcactgaggggccttctgacatgagtctggcctggccccacctcctagttcctcataataaagacagattgcttcttcgcttctcactgaggggccttctgacatgagtctggcctggccccacctccccagtttctcataataaagacagattgcttcttcacttgaatcaagggacctaaaaaaaaaaaa

SEQ ID NO: 14 - DNA, 5743 bp, Homo sapiens
acgtgaccccagcacagctggaggcgggtccccgccctccccagggagcagccggttgaggcgggagcggtgcattgtgggcagaggggcgggggttgggaagatggcggctcccagcctcctcaactggaggcgagtttcttccttcacggggccggtcccccgcgcccggcacggacaccgagcggtggccatccgggagctgatgatcatctttggagggggaaatgagggcatcgcggatgagctgcacgtctacaacacggctacgaatcagtggtttctgccagctgttagaggagatatccctccaggctgtgctgcccatggatttgtctgtgatggtaccagaatattagtatttgggggaatggttgaatatggaagatacagcaatgagttatatgagttacaagcaagtcgttggttatggaaaaaagtgaaaccccatccccctccttctggtttacctccttgtcctcggcttggacatagcttctctttatatggtaacaaatgctatttgtttggtggcctggcaaacgaaagcgaagattcaaacaataatgttcccagatatttaaatgatttttatgagttggagctacagcatggctctggtgttgtgggttggagcattccagtgactaaaggggttgtgccttctccaagagaatcccacacagctgttatatattgcaaaaaagattctggaagtcctaaaatgtatgtttttggtggaatgtgtggtgctcgcctggatgacctatggcagcttgacttagaaactatgtcatggtcaaaaccagaaactaaagggacagtgccacttccacgaagccttcatacagccagtgttataggaaacaagatgtacatttttggtggatgggtcccacataagggggaaaatactgagacttcacctcatgattgtgaatggagatgtaccagttcattttcttacctaaatctggatacaacagagtggaccaccctagtatcagattctcaggaagataaaaaaaattcaagaccaagaccaagagctggccactgtgctgttgcaatcggcactcgattgtatttttggagtggaagagatggctacaaaaaagcactgaatagtcaagtttgctgcaaggatctttggtatcttgatactgagaaaccaccggcaccatctcaagtacagctgatcaaagccactaccaactcctttcatgtcaagtgggatgaagtgtctacagttgagggctatcttttgcagttgagtacagacttgccataccaagctgcatcatcagattcttcagcagcaccaaatatgcaaggagtcaggatggaccctcacagacaaggcagtaataacatcgttcctaacagtatcaatgatacaataaacagcacaaaaactgaacagccagccacaaaagaaacttcaatgaaaaacaaaccagactttaaagcactgacggattctaatgccattttatatccatctttggcatcaaatgcttctaatcataatagtcatgtggtggatatgctaaggaaaaatgaaggtcctcacacttcagcaaatgtaggtgttctaagtagttgcctggatgtaagaacagtaattcctgaaacatctgtatccagtactgtttccagcacacaaactatggtaacccagcagaccattaaaactgaatcatccagtacaaatggggcagttgttaaagatgaaacttcactaacaacattcagtaccaaatctgaagttgatgaaacatatgcactgcctgcaacgaagatcagccgtgtagagacacatgctacagcaacgccgttttctaaagagactccttcaaatccagtggccacagtgaaagcgggagaacgacaatggtgtgatgtgggaatttttaaaaataatacagctttggtgagccagttttatttgctgccaaaagggaagcaaagcatctcaaaggtaggaaatgcagatgtacctgactacagcttgcttaagaaacaagatcttgttccaggcacaggatacagattcagggttgctgcaatcaatggttgtgggataggtcctttcagcaaaatcagtgaatttaaaacttgtattcctggttttcctggagctccttctgcagtcagaatttcaaagaatgttgaaggtatccacctttcctgggaacctccaacctcaccttctggaaatattttggaatattcagcctacttggctatccgcacagcacagatacaagataatccaagtcaacttgtgttcatgaggatttattgtggtcttaagacatcatgtatagtaactgctgggcaacttgcaaatgcacatattgattatacatccaggcctgccattgtgttcaggatatcagcaaagaatgaaaagggatatggaccagctacacaagttcggtggcttcaaggtaacaataagaaagcacctttaaattgaattggtttttttactgaagctattgtgatgatgattatttattagtaactggttatgaagatttgtcatttaaaagagtattctctggctgtatttccagcagttatgaacttgagtttgtaaattgttcttaaaatgtatttgctgaattatagatccaaataaaagaaaagaagcaaagactctctgaaaattagtatatgagttcttccttacagatatagctcttttataaagaaaaacagtgaaattaagataaaagctagaaagctttattaccccaatatcttttataagggctgtgtaacccagtcatcctagagttattgagatgattctggtaactggctgttgtatactatgctgtcttatatagtaaatgttctctacattgtaaatgtcctacctttgagtactccttctatctttattctatatgagcagtgttcctctaacaactgtcttctattggataatatgtgtgatactataataattctaatagttatttgattcctttctttgtttacagttagcacagagcttaagaaattaaaaaagaggcaagtttagaaactgcttttgaacttttattattttccaagaatcatggtaaaatacaacaagaacaatgagttttcttgattcttaccaggccaatatatttgaatatatttttggccaaagcacttaacttatctggccctcattttcctcataacagatgagggagttaaagcagtttatcttttaggtccctttcagcactaaggttttgtgtttttagtagaagtgaagtatgtccctgaacattttcatctgtcttaatggctctaattttgcttttgctaaaattaaaatcttcataattgaattcaaaattaaagtatatgtcctcctattggagaaaaaaacaagcaccttaacaggacacaagttttaaaatagtgttttaaacatttgtaagatttttgtaaatgctctaaatgttttatttttgcattgtttttaccttgaaattgtataaacttttttactatgaaaatataagcattagacagctaactcaggttccattacaaccttaaagatacagataggcattataaacactctgctacaggtaacttgttttaaaccttttaggaatttcatggttccactgcctatttaaatctggaattaatatacagagcaataattgcagaaaagatatttcaaactaagagcttcaataactgcaggggcacaggtctatctttttttataccacataaccttatgccagtttaggttgactgagtagctatgtcctcaaatttcatgtatgataccccccagcaaagatggatttaattgtgcagaattgatatatatgtgtgttccagttactaatttaaagtgtatagaatattttaatatataatttttgtaaagaactgggatttttatactacagtatttgtaattaatgtgtctcactaattaagtttctcttgaagaaaatatgcaaattgttagacacataatgagtggtttccaaactctaaagataaaataaatgcactactatggtgttgttagaataccttttttgtggctgtatgaatctttactctttaaatgtgtactttacaatgtttacaaggagcttctctggtttgttatcccagcaatgcccttggtgagagcttcattctgcatttgtttaattcatatgctttgcttttggcatatgcttgctttttgcactagtagaattttaacttacctcattattatgtttgtaatcatttgtctagcttctgtaatgtatctgatataggctaaacaaatacttaaccaaagttaaaatacatggtaagcaattttgcacatattaaatattagggtttgttgtgtatatgtgtatacttaattataaaagaatatgaatggaaagttatactaaaagtatatgtttcaagcccagttattttaaagatctattgctttaatagaaaaagtttggcttttaaccctttcttctctgcattaattaacaacgtgcatcttgaagcccttctcctgataacaaggaataagttgattttaagcaaaggatagaatatttgtttgagtgtgcacatttatacctgtttagtatttgattcagttaagcctccactcttctggatcaggaggtgattctgaacctgtggttctaacatatgacttaactgtttagagggaagagaagtctttctatactgagggatttgttaaaatcataaatgtttttgccctgagacttagcccagattctattttttttttttttttggcatttgtacattaagcaccagtcatatttatctctgtgataagggattgctgactaaaaaatgccattaagaaatctctgtggctttgacattagtagttaataagagatcttagagctgtgtgctgatgaatagaatgaattggagcaaatagcaagagaagtgcatttagcttcttatgatttgcacattttctaaatgaagatccctttgtgtagcagtaggctgtatcatgcagatatttttcataggatttgcactgagttaaatgactttctttactcttcccagtcaggtaagcagttcaaatataacaagactttgtgccagtcctcttctgtgttctgcaactctggctctcataagtagtaaaaatatttttgatgttgcatgctctcagaattatgttttctaaaatatcatattggcttcattttagtttaataaacttctttggcaacttaattagacatagggtatatataaactcaattttatctgcattttttaaaaaattgaaataatctgattgcatgtatgaaggactcctgcctcattacccaattacataaatacaattatctataggaaggtgaaattgttgatctattatttcaaagacttgtagactagcaaagattcattctgattagaaataaggtctatagctaaagatatttttattttaaaaaattacagcccttgtaacaaaaatcatttgtaacattggatatgaagacattattagtatttaaaattattgtagtcatttgatcataaatgaagtagcttgcaaaatagacttggtctttgaccctttaaggtgtaactgactgactgttgtgtttttcaagactgattttaggcagttttatgtatcgtgtaccaatgatatgtaaaataaagcaccttttctactataaacaaaaaaaaaaaa

SEQ ID NO: 15 - DNA, 941 bp, Homo sapiens
cgcctccctcgctccacgcgcgcccggactcggcggccaggcttgcgcgcggttcccctcccggtgggcggattcctgggcaagatgaagtgggtgtgggcgctcttgctgttggcggcgctgggcagcggccgcgcggagcgcgactgccgagtgagcagcttccgagtcaaggagaacttcgacaaggctcgcttctctgggacctggtacgccatggccaagaaggaccccgagggcctctttctgcaggacaacatcgtcgcggagttctccgtggacgagaccggccagatgagcgccacagccaagggccgagtccgtcttttgaataactgggacgtgtgcgcagacatggtgggcaccttcacagacaccgaggaccctgccaagttcaagatgaagtactggggcgtagcctcctttctccagaaaggaaatgatgaccactggatcgtcgacacagactacgacacgtatgccgtgcagtactcctgccgcctcctgaacctcgatggcacctgtgctgacagctactccttcgtgttttcccgggaccccaacggcctgcccccagaagcgcagaagattgtaaggcagcggcaggaggagctgtgcctggccaggcagtacaggctgatcgtccacaacggttactgcgatggcagatcagaaagaaaccttttgtagcaatatcaagaatctagtttcatctgagaacttctgattagctctcagtcttcagctctatttatcttaggagtttaatttgcccttctctccccatcttccctcagttcccataaaaccttcattacacataaagatacacgtgggggtcagtgaatctgcttgcctttcctgaaagtttctggggcttaagattccagactctgattcattaaactatagtcacccgtgtcctgtgaaaaaaaaaaaaa

SEQ ID NO: 16 - DNA, 7074 bp, Homo sapiens
catttctacttcatatgatcattttaattgtatttacctcatcttaccaaaaagatcatggttgaatcttgcctactcacgtttagggcctttttttggtggattgctttaataaagatggatctgtcagaccttggagaagccgcagccttcctcagaagaagtgaagctgagctgcttctactacaggccacagccttggatgggaagaagaaatgctggattcctgatggtgagaatgcttatatcgaggctgaggtaaaagggagtgaagatgatggaacagtaattgttgagacagcagatggagagagtctgagcataaaggaggacaaaatccagcagatgaatcctccagagtttgaaatgattgaagacatggcaatgctgactcacctcaatgaggcatccgtgctgcataccctgaagcggcgctatggccagtggatgatctatacatattcaggtctcttctgtgtgaccataaacccttacaaatggcttcccgtgtatcagaaagaagtcatggccgcctacaaagggaagaggcgatcagaggctccccctcacatctttgctgttgccaataacgcctttcaggacatgcttcacaatcgagaaaatcagtctatactcttcacaggagaatctggtgctggaaagactgtgaacagcaaacatattatccagtattttgccaccatagcagccatgattgaatccaggaaaaagcagggggcgttagaagatcaaatcatgcaagcgaatactatcttggaagcatttggaaatgctaaaaccctgagaaatgacaactcctctcgttttggcaaattcatcaggatgcactttggtgccagaggcatgctgtcatctgtggacattgatatctatttgcttgaaaagtccagggtgattttccagcaggctggagagaggaactaccacatattctatcaaattctatctggacaaaaagagcttcatgacctgctcctggtatctgcaaatccctcagacttccacttttgctcctgtggagcagttactgtggagagcttggatgatgctgaagaattgctggccacagaacaagccatggacatcttgggctttcttcctgatgagaagtatggatgctataaactcactggagccatcatgcactttggaaatatgaaatttaaacagaaacctagagaagagcaactggaagcagatggcacagaaaatgctgacaaagctgctttcctcatgggcattaactcctctgagttggtaaagtgcttgatccatcctagaatcaaagttggtaacgaatatgttaccagaggtcaaactatagaacaggtaacctgtgctgtcggtgccctgtccaagtcaatgtatgaaaggatgtttaagtggctagtggcacggatcaacagggccctggatgccaagctgtcaaggcagttcttcattggcattcttgacatcactggttttgaaatccttgagtataatagccttgagcaactttgcattaattttaccaatgaaaaattacaacaattcttcaattggcacatgtttgttctggagcaagaggaatataagaaagaaagcattgaatgggtgtctattggctttggtctggatttgcaagcttgcatagatctcattgagaagccaatgggcatcctttccatccttgaagaagagtgtatgtttcctaaggctacagacctgactttcaagaccaaactctttgacaaccattttggaaagtcggttcatctccagaagcccaagcctgataagaagaaatttgaagctcattttgaacttgtccattatgcaggagtggtaccttataatatcagtggttggctggaaaagaacaaagacctccttaatgaaacagtggtagctgtatttcagaagtcttccaacagactcctggcgagcctttttgaaaattacatgagtactgacagtgctataccatttggggagaagaaacgaaagaaaggagcttcattccaaacggttgcatctctgcataaagaaaacctgaataaattgatgactaatctgaaatcaacagcacctcattttgtgagatgcataaatcccaatgtgaacaaaataccaggtatactggacccttacttggttctacagcagttgcgctgtaatggtgtcttggaagggactaggatatgccgtgaaggttttccaaaccgactgcagtatgctgattttaaacaaaggtactgcattctgaatccaaggacctttccaaagagcaagtttgtgagcagcagaaaagcagctgaagaattacttggctccttggagatagaccatacccagtaccgatttggaatcactaaggtgttttttaaagctgggtttctgggccaactggaagcaataagagatgagagactatctaaagtcttcacattgttccaagccagagcacagggcaaactgatgcgaatcaaattccagaagattctggaagaaagggatgcacttattttgatccaatggaacataagagctttcatggctgtgaagaactggccctggatgaggctcttcttcaagatcaagcctcttgttaaatcttcagaagtaggagaagaagtagctggactgaaggaagagtgtgcacaattacagaaagccttggagaaatcagagtttcagagggaggaactgaaagcaaagcaagtatccctcactcaggaaaaaaatgacctgattcttcagcttcaggctgagcaagagacactggcaaatgttgaagagcagtgcgagtggctgattaaatccaagatccagctggaggccagagtaaaggagctgtcggagagggtggaggaagaagaggagataaattctgagctgactgccagggggcggaaactcgaagatgaatgttttgagttgaagaaagaaatcgatgacctggaaacaatgttggtgaagtcagagaaggagaagcgtactacagagcacaaggtcaagaacttgactgaggaagtagagtttctaaatgaggatatcagcaaacttaacagagcagccaaggttgtgcaggaggcccatcagcagaccctggatgacctgcacatggaggaggagaagctcagcagcctgagcaaagcaaatctgaagctggaacagcaagttgatgagcttgagggtgcccttgagcaggagagaaaagcgagaatgaactgtgaaagggaactgcacaaactggagggcaatttaaagctgaatcgggaaagtatggagaacctggaaagcagccagcgacacctggcagaagagctgaggaaaaaagaattagaattgagtcagatgaattcaaaagtggagaatgagaaaggcctggtagctcagcttcagaagacggttaaagagcttcagactcaaataaaggatttgaaagagaaactagaagctgaaaggaccactcgagccaagatggaaagggagagagctgacctcacccaagacctggctgacttgaatgagaggctggaggaggtaggaggatccagtttggctcagctggaaataactaagaaacaggaaaccaaattccagaagctgcaccgagacatggaagaggccactctgcactttgagacaacttctgcatctttgaagaagagacatgcagacagcctggctgagctcgagggccaggtagaaaatctacagcaggtcaagcagaaactggaaaaagacaagagtgacttgcagctagaagtagatgacctcctgacccgtgttgagcagatgacaagagctaaggcaaatgctgagaaactctgtactctatatgaagagcgcttgcatgaagcaactgcaaagctagataaggtgactcagttggcaaatgacctggcagcacaaaagacaaagctgtggagtgagagtggcgagttcctacggaggcttgaagagaaggaggctctgataaaccaactttccagggaaaagagcaacttcactcggcagattgaagacctgagagggcagctggaaaaggagaccaaatcccagagtgccctggcccatgccctgcagaaggctcagcgtgactgtgaccttctacgagagcagtatgaggaagaacaagaggtcaaggctgagctgcaccggaccttatccaaagtcaatgctgaaatggtgcaatggagaatgaagtatgaaaacaatgtcatccagagaacagaagacttggaggatgccaagaaggaactggcaattagattgcaggaggcagccgaagccatgggggtggccaatgccagaaatgcctccttggagagagccaggcaccagctgcagctggagctcggggacgccctgtctgacctcgggaaggtccgctctgcagcagccaggctggaccagaagcagctgcagtctggcaaggcccttgccgactggaagcagaagcacgaggagtcccaggcgttgctggatgcctctcagaaggaagttcaggctctcagtacagagctcctcaagctcaagaacacctatgaggagagcatcgtgggccaggagacactcaggagggagaacaagaacctccaagaagagatttctaatctgacaaaccaggttagagaagggaccaagaacttaactgaaatggaaaaggtcaagaaactaattgaagaagagaagacagaagtccaggtgacactggaagaaacagagggagccctggaacgtaatgaaagcaagattcttcatttccagcttgaactcttggaagctaaagcagaacttgaaagaaagctttcagagaaagatgaagaaatagaaaattttaggaggaagcagcagtgtaccattgactccctgcagtctagtctggattctgaagctaagagcagaattgaggttacccggctgaagaagaagatggaagaggacctcaatgagatggaactccagcttagctgtgccaaccggcaggtgtcagaagcaaccaaatccctgggccagcttcagattcaaatcaaggaccttcaaatgcagctggatgacagcacacaactgaacagtgatctgaaggagcaggtggctgtggctgagcggcgcaactctcttcttcagtctgaactagaggatctaaggtccctgcaagagcagacagagcgtggccgcaggctgtcagaagaagagctcctggaagcaacagaaagaatcaatcttttctatacccagaacacaagcctcctcagccagaagaagaaactggaggctgatgttgcccggatgcagaaagaagctgaagaggtggtgcaggagtgtcaaaatgcagaagagaaggccaagaaggcagccattgaggcagcaaacttgtcagaagaactgaagaagaagcaagacaccattgcccacttggaaaggacaagagaaaatatggagcagacaattacagacttacagaaaaggctggctgaagctgaacagatggccctgatggggagtagaaagcaaatccagaaactagaatccagggttcgtgaactggaaggtgaactggagggtgaaatccgtcgcagtgcagaggcccagaggggagcccgcagacttgagcgatgcatcaaagagctgacctatcaggcagaggaagacaagaagaatctgagcaggatgcaaactcagatggataaacttcagctaaaagtgcaaaattacaagcagcaagtcgaggtggcggaaacacaagccaatcaatacctttccaagtataagaaacagcaacatgagttgaatgaagtgaaggaaagggcagaggtggcagaatctcaagtcaataaactcaaaattaaagcaagagagtttgggaaaaaggttcaagaagaatagcatcccctgctttgaaaggacaacagctggagaagtacaaggaaggtgctgtttcatggccaaaaacttaggttgcatggaaacatttttaaaaacatgtttaaattgcttttcacaccatataaacaaggcaattagaaaaataattaaagggaatatcattgcttccacagttaatggggattttttgatcctcaaatgcaagtaaactaccttctaatgcttcacatgacagattaaataaatggaagaaccttttcaattctgatgtttaaaaaatgaataaatacttgatcctttgtccatatttcctcttaatgggtaggactcatagaagatgtccttagaccagtcacgcttcatggggactagggcatgttggtgaatggtttttactaaagttaggcaactttggcttgattcacccctaaatctatgaatgtatattgtgagccagcagtggtagaatggaagtcataagatctcctcagcattgtgatataaatatagctatagttaggcaatttgaacatgtaggcaaaactctcctaatcaacacacatgtaggctatatgctggtacatgctttaaacatggaggtaaccccacacgagacattcagtgacaggtaatatgctggatttgtgcagtaccgatttggctcagctggaggaacatgtcccggaattcctctctctcatggttctggattggagtaggtcataaagaaatttgcattaatttgatcagcagctattttatactcataaggtcagtgtacagacccaagcatggtgacagcttgaaaatatgactccaggccaaaaaggggagctagaagagaccagagacagctccctggacccagagctcttccagctcctgccagcctcctccttcagctttgcaaagtactggccaggtgtgtgtgcagctccatggcaaccagcatcagcttttcctgagatcacacacagcattgcagtggaggccgtgagacagacatgggttctgtttgttcttatggacttcccttcatccttgctgcattcacagtcgacctacagtgacttcaggcccagaaccagatgcagagggaacagcctggcctagacttctccaccagcacccacaattgtgtaaggctgaagctctataataaatctttattctgtc

(19) United States
(12) Patent Application Publication
(10) Pub. No.: US 2011/0124511 A1
(43) Pub. Date: May 26, 2011

(54) Gene Expression Profile-Facilitated In Vitro Fertilization

(76) Inventors: William B. Schoolcraft, Denver, CO (US); Mandy Katz-Jaffe, Denver, CO (US)

(21) Appl. No.: 12/906,049

(22) Filed: Oct. 15, 2010

Related U.S. Application Data
(60) Provisional application No. 61/252,134, filed on Oct. 15, 2009.

(51) Int. Cl. C40B 30/00; C12M 1/34; C40B 60/12; C12Q 1/68

(52) U.S. Cl. 506/7; 435/287.2; 506/39; 435/6.1; 435/6.12

(57) ABSTRACT

Gene expression profiling improves the pregnancy success rate of in vitro fertilization processes, while reducing the risk of multiple births.

### RELATED APPLICATION

This application claims priority to U.S. provisional application Ser. No. 61/252,134 filed Oct. 15, 2009, which is hereby incorporated by reference.

### SEQUENCE LISTING

### Background

1. Field of the Invention

The present disclosure relates to the field of in vitro fertilization (IVF), which is a process by which mammalian egg cells are fertilized by sperm outside the body of a mammal. More particularly, a molecular diagnostic test involving expression profile of one or more genes is used to enhance the pregnancy success rate when the fertilized egg is implanted into the patient's uterus.

2. Description of the Related Art

In successful use since the late 1970's, IVF is an infertility treatment often employed after failure of other assisted reproductive technology methods. IVF overcomes female infertility due to problems of the fallopian tube or endometriosis. IVF may also assist in resolving male infertility due to problems with sperm quality or quantity. In general, IVF offers infertile couples a chance to have a biologically related child. The IVF process involves hormonally controlling the ovulatory process, removing eggs (termed ova) from the woman's ovaries and permitting the sperm to fertilize the eggs in a fluid medium. The fertilized egg (termed embryo) is then transferred to the patient's uterus with the intent of establishing a successful pregnancy. Ideally, IVF candidates can provide healthy eggs, sperm that can fertilize, and a uterus able to maintain a pregnancy. Due to the costs of the procedure, IVF is generally attempted only after less expensive options fail.

FIG. 1A shows a schematic for mammalian blastocyst implantation. A blastocyst, surrounded by zona pellucida and containing a trophectoderm hull, approaches the endometrial wall likely due to paracrine signaling. Additionally, this paracrine signaling likely induces the blastocyst hatching from the zona pellucida. Upon apposition of the blastocyst to the endometrial wall, biochemical signaling occurs involving signaling molecules such as Leukemia inhibitor factor (LIF). For example, during this time LIF is produced by the endometrial wall and the LIF receptor is expressed by the blastocyst. Upon blastocyst adhesion to the endometrial wall, the endometrial wall begins production of LIF receptor and the soluble protein, gp130. The adhesion between the blastocyst and the endometrium causes the trophoblast cells to differentiate into inner cytotrophoblast and outer syncytiotrophoblast layers. In general, the interaction between the blastocyst and the endometrium is a function of both a receptive endometrial environment and a healthy blastocyst. A blastocyst that will not implant or an endometrium that will not sustain growth and differentiation will result in a spontaneous abortion. The prior art teaches very little about embryo's role in the events leading to the attachment of a viable blastocyst to a receptive uterine luminal epithelium.

IVF treatment begins with administration of hormonal medications to stimulate ovarian follicle production. Hormonal treatment cycles typically start on the third day of menstruation, constituting about ten days of injections. These injections consist of protein hormones, termed gonadotropins, utilized under close monitoring. This monitoring frequently involves evaluating the estradiol hormone levels and ovarian follicular growth. The prevention of spontaneous ovulation involves utilization of other hormones such as GnRH antagonists or GnRH agonists that block the natural surge of luteinizing hormone.

With adequate follicular maturation, administration of human chorionic gonadotropin hormone causes ovulation approximately 42 hours after the administration. However, the egg retrieval procedure takes place just prior to ovulation, in order to recover the eggs from the ovary. The egg retrieval proceeds using a transvaginal technique involving an ultrasound-guided needle that pierces the vaginal wall to reach the ovaries. After recovery of the follicles through the needle, the follicular fluid is provided to the IVF laboratory to identify eggs. Typically, the procedure retrieves between 10 and 30 eggs. The retrieval procedure takes approximately 20 minutes and is usually done under conscious sedation or general anesthesia.

For IVF, the fertilization of the egg (termed insemination) proceeds in the laboratory where the identified eggs and semen are usually incubated together at a ratio of about 75,000:1 in a culture media for about 18 hours. The confirmation of fertilization proceeds by monitoring the eggs for cell division. For instance, a fertilized egg shows two pronuclei.

Selected embryos are transferred to the patient's uterus through a thin, plastic catheter, which goes through the vagina and cervix. Typically, transfer or implantation of 6-8 cell stage embryos to the uterus occurs three days after embryo retrieval. In many American and Australian programs, embryos are placed into an extended culture system with a transfer done at the blastocyst stage at around five days post-retrieval. Blastocyst stage transfers often result in higher pregnancy rates. Additionally, embryonic cryopreservation, or the storage of embryos in a frozen state, is feasible until uterine transfer. For example, the first term pregnancy derived from a frozen human embryo was reported in 1984. Since then, estimations reveal that births of IVF babies derived from frozen embryos stored in liquid nitrogen exceed 350,000.

The process for selecting embryos for transfer often involves grading methods developed in individual laboratories to judge oocyte and embryo quality. An arbitrary embryo score involving the number and quality of embryos may reveal the probability of pregnancy success after transfer. For example, the embryologist grades the embryos using morphological qualities including the number of cells, clearness of cytoplasm, evenness of growth and degree of fragmentation. However, embryo selection based on morphological qualities is not precise. Often, several embryos selected for these general qualities are implanted to improve the chance of pregnancy. The number of embryos transferred depends upon the number available, the age of the woman and other health and diagnostic factors. In countries such as the United Kingdom, Australia and New Zealand, a maximum of two embryos are transferred except in unusual circumstances. The United Kingdom permits a maximum transfer of three embryos for women over 40. In contrast, the United States permits the transfer of multiple embryos based upon the individual fertility diagnoses of younger women. The limitations on the number of transferred embryos occur because most clinics and country regulatory bodies seek to minimize the risk of multiple pregnancies.

Multiple pregnancies, related to the practice of transferring multiple embryos at embryo transfer, is a major complication of IVF. In general, multiple pregnancies, specifically, more than twins, should be avoided because of the associated maternal and fetal risks. Multiple births are related to increased risk of pregnancy loss, obstetrical complications, prematurity, and neonatal morbidity with the potential for long term damage. Some countries implemented strict limits on the number of transferred embryos to reduce the risk of high-order multiples (e.g., triplets or more). However, these limitations are not universally followed or accepted.

Although the success rates of IVF are rising, the overall rates are still relatively low. For example, Canadian clinics reported an average pregnancy rate of 35% for one cycle, but a live birth rate of only 27% in 2006. Moreover, success rates vary with the age of the mother if donor eggs are not used. Currently, IVF attempts in multiple cycles result in increased cumulative live birth rates. Depending on the demographic group, one study reported 45% to 53% for three attempts, and 51% to 71% for six attempts.

As shown in FIG. 2, two previously published studies—one in the bovine and the other in the human-investigated a hypothesis that viable embryos possess specific gene expression profiles that characterize their ability to develop, and successfully implant. In general, both of these studies analyzed pooled trophectoderm (TE) biopsies and observed that a specific TE gene expression profile appeared to correlate with implantation potential. In particular, the bovine study utilized pooled bovine trophectoderm biopsies to investigate pregnancy outcome by analyzing gene expression differences using microarray technology. In this bovine study, a total of 52 bovine genes were identified when comparing no pregnancy with calf delivery and a total of 58 bovine genes were identified when comparing resorbed embryos with calf delivery. The previously published study involving human embryos utilized comparative microarray analysis of cDNA from pooled ‘viable’ and ‘non-viable’ TE samples and identified over 7000 transcripts expressed exclusively in ‘viable’ blastocysts. These results support that a considerable portion of implantation failure is attributed to the embryo rather than the uterus. For example, singleton pregnancies may result from the transfer of multiple embryos of equivalent morphology.

### SUMMARY

The present disclosure overcomes the problems outlined above by improving pregnancy success rates with lower incidence of multiple births.

A system for enhancing the pregnancy success rate of in vitro fertilization includes an analytical system that determines the gene expression profile of a blastocyst to identify modulation of one or more genes implicated in implantation success. On the basis of this molecular diagnostic gene expression profile, the system recommends whether to implant the blastocyst, which may be conditionally implanted on the basis of this recommendation. In one aspect, the genetic material of a blastocyst obtained from trophoblast cells and the gene expression profile is further analyzed via quantitative real time PCR. In another aspect, the gene expression profile of a blastocyst is obtained using a panel of PCR primers designed to evaluate the developmental competence and implantation potential of the embryo.

In one embodiment, a system for enhancing the success rate of a pregnancy resulting from an in vitro fertilization is disclosed. The system may contain means for determining the expression profile of at least one gene in at least one cell obtained from a blastocyst. The system may further include means for determining the probability of success of a pregnancy that would result from the blastocyst based upon the expression profile.

In another embodiment, the system may include genetic material(s) obtained from the blastocyst, such as DNA or RNA obtained from the blastocyst. In one aspect, the system may include means for determining whether or not to implant the blastocyst based on the probability of a success of pregnancy that result from implantation of the blastocyst. In another aspect, the system may further include means for recommending whether or not to implant the blastocyst based on said determination of said probability of success of pregnancy.

In another embodiment, it is disclosed a method for increasing the probability of a successful pregnancy resulting from an in vitro fertilization, which includes the steps of (a) determining the expression profile of at least one gene in a cell from a blastocyst resulting from the in vitro fertilization; and (b) determining the probability of a successful pregnancy resulting from said blastocyst. The probability of a successful pregnancy may be determined based upon the expression profile of said at least one gene.

In another aspect, the disclosed method may include a step of determining whether to implant the blastocyst based on the determination of probability of a successful pregnancy as performed in step (b). In another aspect, the disclosed method may include a step of recommending whether to implant said blastocyst based on said probability of successful pregnancy determined in step (b).

The expression profile of the one or more cell from the blastocyst may be determined by quantitative polymerase chain reaction (PCR) or by microarray analysis, such as gene chip analysis. In one aspect, the expression profile of at least one gene may be determined, wherein the at least one gene is implicated in at least one function of implantation, absorption or development of a blastocyst. More specifically, the at least one gene may be any one or more of the polynucleotides of SEQ ID Nos 2-11 or SEQ ID Nos 13-16.

In one embodiment, the at least one gene may be one or more polynucleotides of SEQ ID Nos 3-10, where downregulation of the expression of any one of these genes as compared to a normalized control may indicate a decreased probability of a successful pregnancy resulting from the blastocyst. Downregulation may be reduction by at least 20%, 30%, or more preferably, by 50%, or even more preferably, by at least 80%.

In another aspect, the at least one gene may be the polynucleotide of SEQ ID No 11 where upregulation of the expression of the gene as compared to a normalized control may indicate a decreased probability of a successful pregnancy resulting from the blastocyst. Upregulation may be an increase by at least 20%, 30%, or more preferably, by 50%, or even more preferably, by at least 100%. The housekeeping gene encoding glyceraldehyde-3-p-dehydrogenase (GAPDH) may be used as the normalized control. For purpose of this disclosure, a successful pregnancy is one that will ultimately result in the development of a live baby under normal circumstances.

### BRIEF DESCRIPTION OF THE FIGURES

FIG. 1A is a diagram illustrating the process blastocyst implantation.

FIG. 1B is a schematic drawing showing in more details the structure of the blastocyst 100 and the Trophectoderm 102.

FIG. 2 is a diagram illustrating a hypothesis that viable embryos possess specific gene expression profiles.

FIG. 3 is a table that illustrates the success rates from donor oocyte IVF cycles.

FIG. 4 is a diagram illustrating the process for murine blastocyst retrieval, culture and biopsy.

FIG. 5 is a flow chart showing the process of gene expression analysis to measure the expression profile of one or more genes implicated in implantation success of a blastocyst.

FIG. 6 is a picture that demonstrates the surgical removal of mouse uterine horns.

FIG. 7 is a flow chart illustrating the overall process of TE gene expression analysis by quantitative real-time PCR

FIG. 8 is a histogram that displays changes in gene expression for B3gnt5, Cdx2 and Slc7a5 in murine embryos as analyzed by microarray technology.

FIG. 9 is a histogram that displays changes in gene expression for Eomes, Wnt3a and Wnt5a in murine embryos as analyzed by microarray technology.

FIG. 10 is a diagram that displays whole transcriptome analysis of murine embryos.

FIG. 11 is a diagram that displays whole transcriptome analysis of murine embryos with an emphasis on the predominant gene ontology biological processes.

FIG. 12 shows the Complement & Coagulation Cascade.

FIG. 13 is a histogram that displays the changes in gene expression for Cdx2 in murine embryos as analyzed by real-time PCR.

FIG. 14 is a histogram that displays the changes in gene expression for Igf2 in murine embryos as analyzed by real-time PCR.

FIG. 15 is a histogram that displays the changes in gene expression for Ascl2 in murine embryos as analyzed by real-time PCR.

FIG. 16 is a histogram that displays the changes in gene expression for Sh2b3 in murine embryos as analyzed by real-time PCR.

FIG. 17 is a flow chart that illustrates individual TE gene expression profiling directly predicting ongoing healthy fetal development.

FIG. 18 is a diagram illustrating the process of determining a gene expression profile of a blastocyst.

FIG. 19 shows the sequence of a number of murine genes implicated in implantation or other embryonic developmental processes, as well as the sequence of the housekeeping mGAPDH gene, used as a normalized control in this disclosure.

FIG. 20 shows the sequence of a number of human genes implicated in implantation or other embryonic developmental processes, as well as the sequence of the housekeeping hGAPDH gene, used as a normalized control in this disclosure.

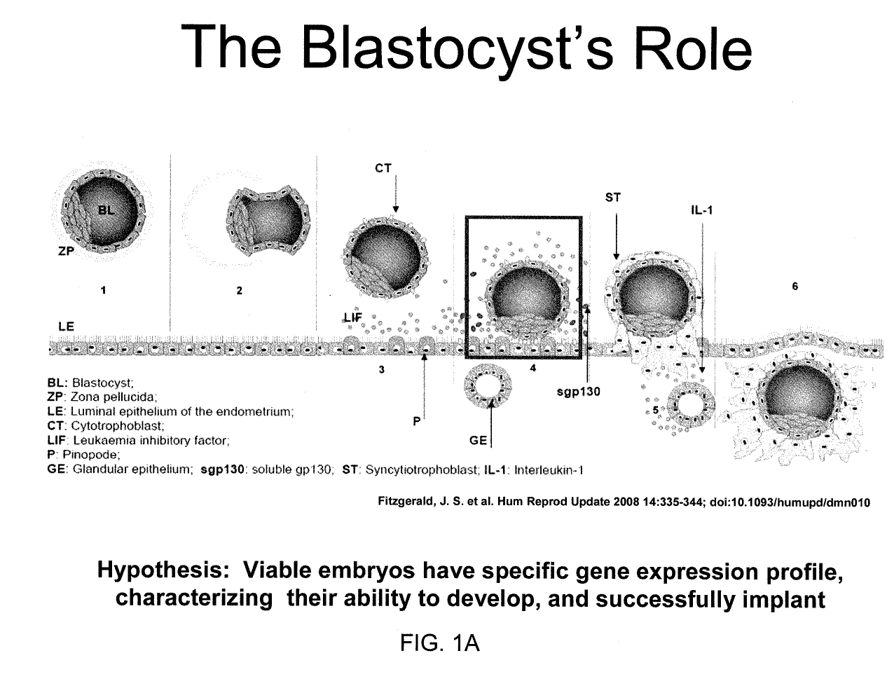

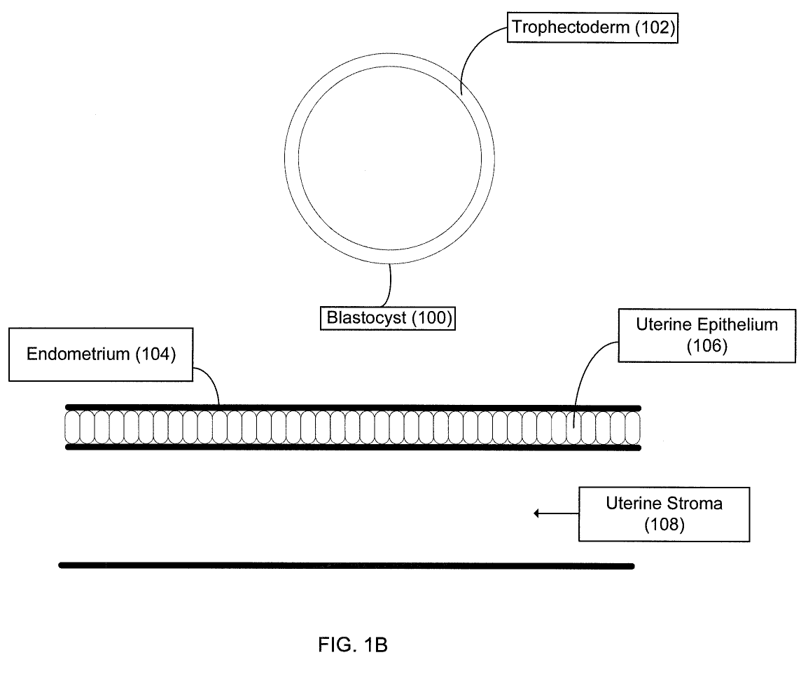

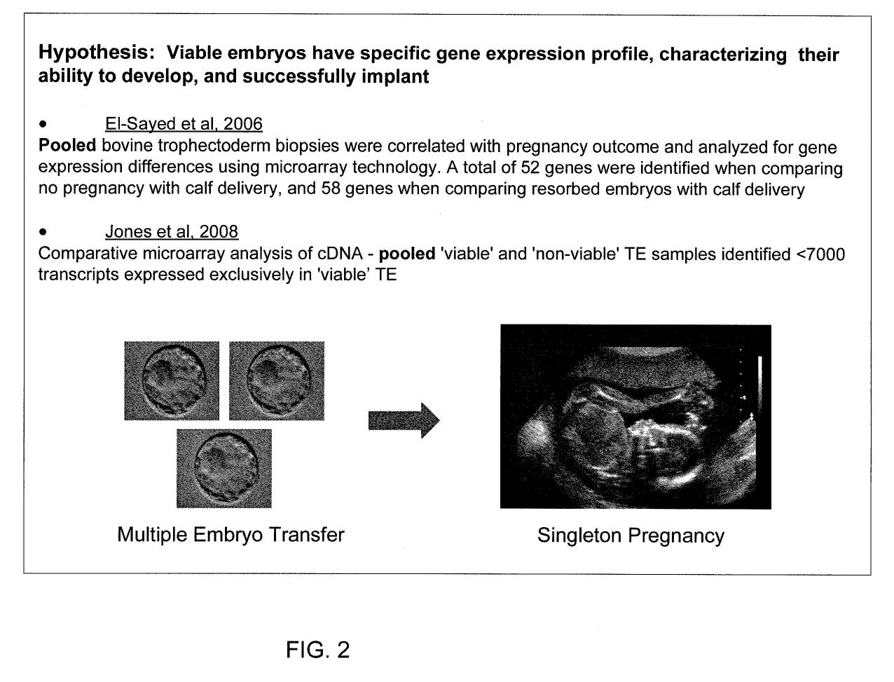

### DETAILED DESCRIPTION

Mammalian embryo implantation is a complex and intricate process involving numerous biological changes at both the embryo and endometrial level. Despite progressively improving IVF pregnancy rates, the majority of transferred human embryos result in implantation failure. Numerous factors are believed to contribute to implantation failure, including embryo chromosome aneuploidies related to advanced maternal age, and maternal factors such as failure of the endometrium to respond through hormone regulation.

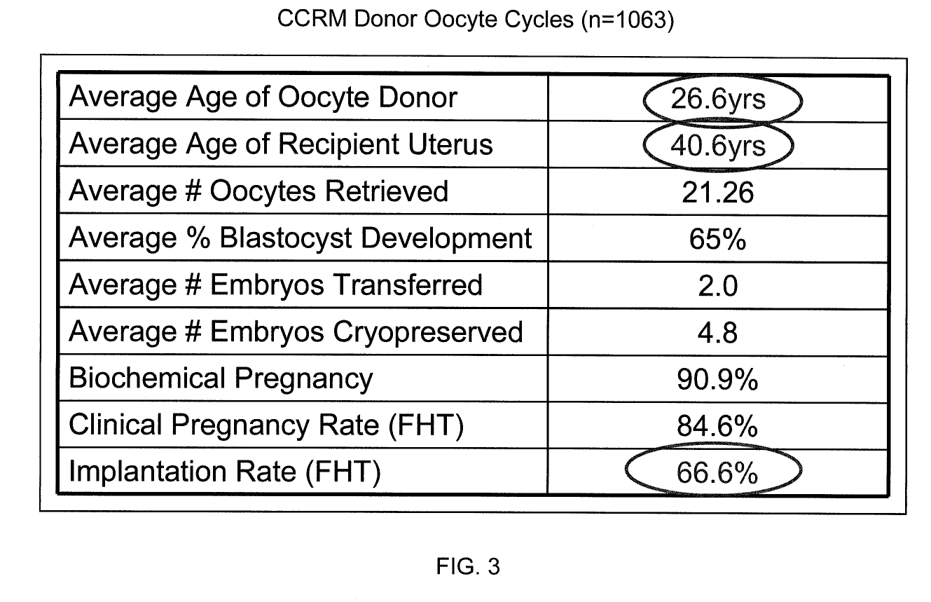

FIG. 3 highlights the importance of embryo developmental competence by showing high success rates from donor oocyte IVF cycles using young reproductive age oocytes transferred to an advanced maternal age endometrium. The data is provided for a Colorado clinic from 2004-2009 including outcomes for over 1000 donor oocyte cycles. These results demonstrate a 66.6% implantation rate for IVF recipients with an average endometrial age of 40.6 years and donor ooctyes with an average age of 26.6 years.

The following descriptions will show and describe, by way of non-limiting example, a process for improving pregnancy success rates with lower incidence of multiple births. The following examples, describing either human or mouse samples, describe the process for evaluating gene expression profiles of TE cells extracted from blastocyst samples to ultimately provide a valuable implantation recommendation. In particular, the a panel of individual genes, each selected for significant developmental competence and implantation potential, are monitored utilizing quantitative real-time PCR reaction.

### Example 1

### Relating Gene Expression Profile to Implantation Success or Failure in a Murine Model

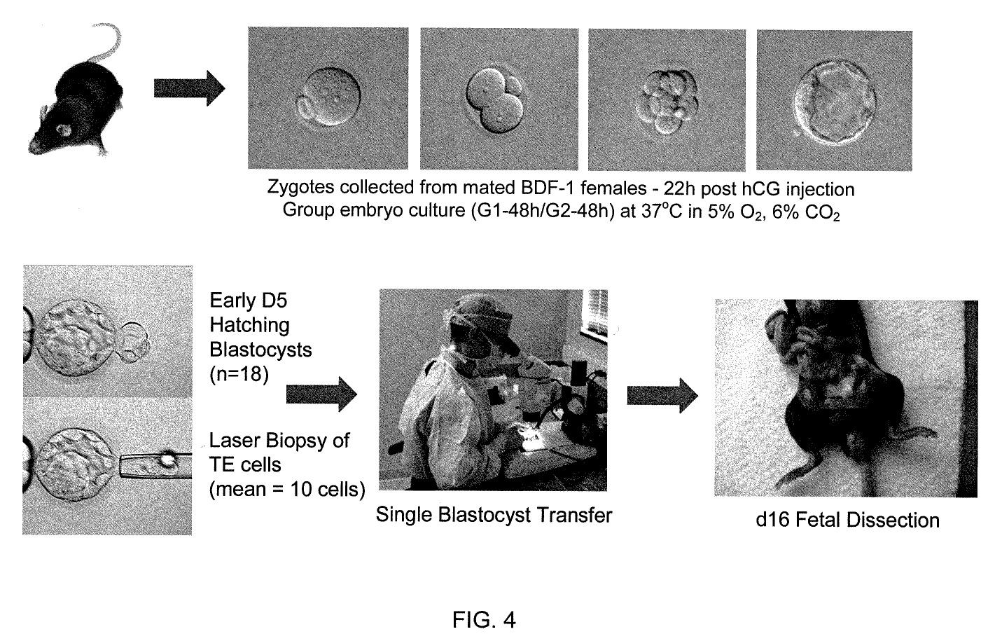

As illustrated in FIG. 4, female BDF-1 mice were superovulated via gonadotrophin injections prior to matings. At 22 hours post-injection, the zygotes were collected and group cultured in microdrops of G1 under an oil overlay at 37° C., 5% O₂ and 6% CO₂ for 48 hours, after which they were transferred into G2 blastocyst stage culture media for a further 48 hours. The identification of hatching blastocysts for biopsy occurred early on the fifth day. Using a laser, herniating TE cells were biopsied to obtain material for gene expression analysis. A single blastocyst, with a known gene expression profile, was implanted into a mouse and further permitted to develop for sixteen days. On the sixteenth day, fetal dissection occurred to investigate the embryo implantation success.

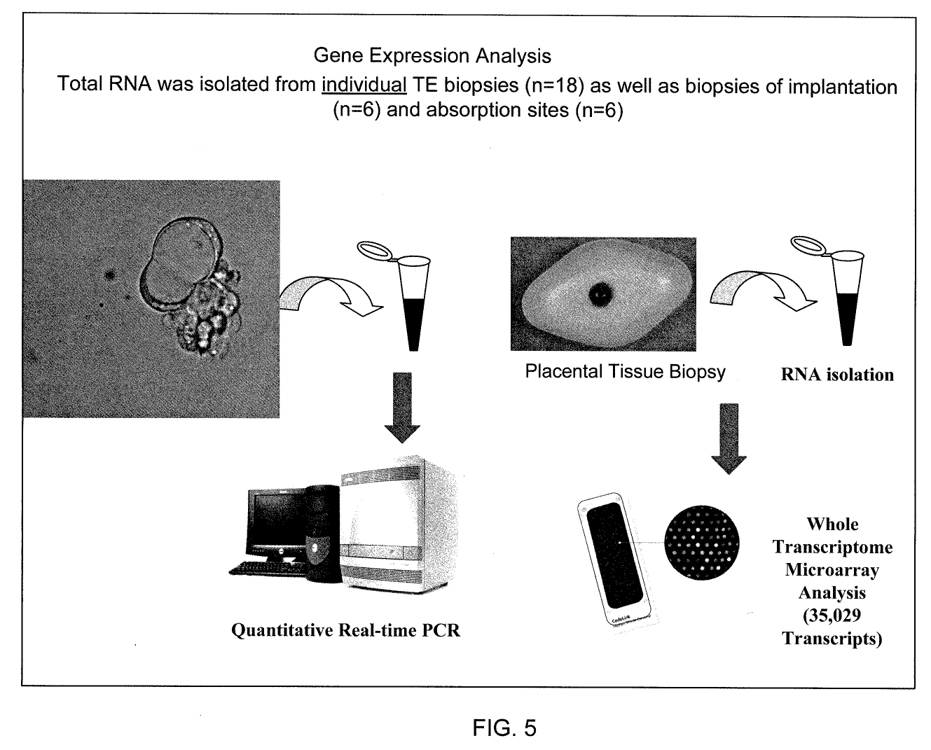

FIG. 5 demonstrates the process of gene expression analysis to measure the expression profile of one or more genes implicated in implantation success of a blastocyst. First, total RNA is isolated from biopsied blastocyst TE cells or from placental tissue using RNA isolation protocols intended for samples containing limited quantities of RNA. In one embodiment, RNA isolation approach proceeds using the Arcturus® PicoPure® RNA isolation kit (Molecular Devices, Sunnyvale, Calif.) via the manufacturer's recommended protocols. Next, the total isolated RNA is quantitatively converted to cDNA using a reverse transcription reaction. In one example, the reverse transcription occurs using High Capacity cDNA Reverse Transcription Kit (Applied Biosystem, Foster City, Calif.). Following the first reverse transcription reaction, the cDNA is further amplified. In one embodiment, this cDNA amplification occurs using TaqMan® PreAmp Master Mix (Applied Biosystems, Foster City, Calif.) that permits the performance of 200 real-time PCR reactions from as little as 1 ng of cDNA.

As shown in FIG. 5, upon generation of adequate cDNA, gene expression analysis is performed using either quantitative real-time PCR or whole transcriptome microarray analysis. For microarray investigations, whole transciptome analysis is performed with Codelink™ Whole Genome Mouse Bioarrays (Applied Microarrays, Tempe, Ariz.) that contains over 35,000 transcripts using the manufacturer's recommended protocols. For quantitative real-time PCR, the expression profiles of a gene panel are analyzed using a real-time PCR thermocycler (Applied Biosystems, Foster City, Calif.). In this process, the gene panel consists of one or more genes implicated in implantation success of a blastocyst. For example, a panel of individual genes, each selected for significant developmental competence and implantation potential, are monitored during the reaction using primers designed for each gene of interest. For selection and optimization of PCR primers, see Sambrook and Maniatis, Molecular Cloning: A Laboratory Manual, 3^{rd} ed., Cold Spring Harbor; see also Bashir et al., J Comput Biol. 2010 March; 17(3):369-81, both of which are hereby incorporated by reference into this disclosure.

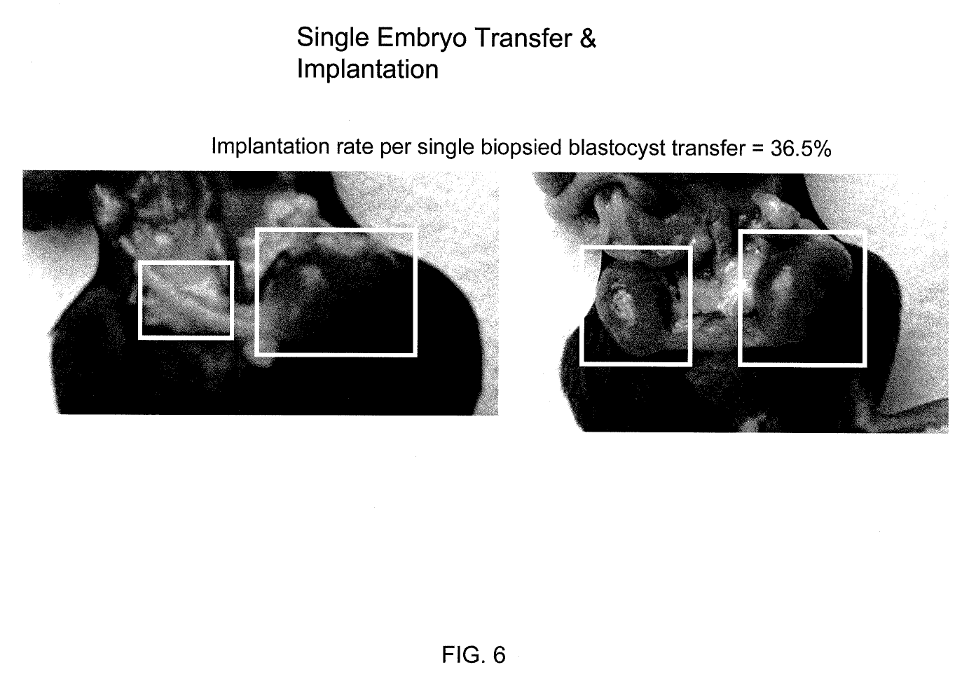

As illustrated in FIG. 6, the surgical removal of uterine horns from each individual mouse confirmed either the presence of a fetus, an implantation site or a complete lack of implantation on day 16 of fetal development. Individual mice with uterine horns displaying either a positive implantation site and subsequent absence of a fetus or an absorption site and subsequent absence of a fetus were considered negative for implantation success. In many instances, single blastocyst implantation in both uterine horns was observed, as shown in FIG. 6. The implantation rate per single biopsied blastocyst transfer per uterine horn was 36.5%. This is a comparable implantation rate as that observed in human IVF. However, typical murine litter sizes exist from between 12-15.

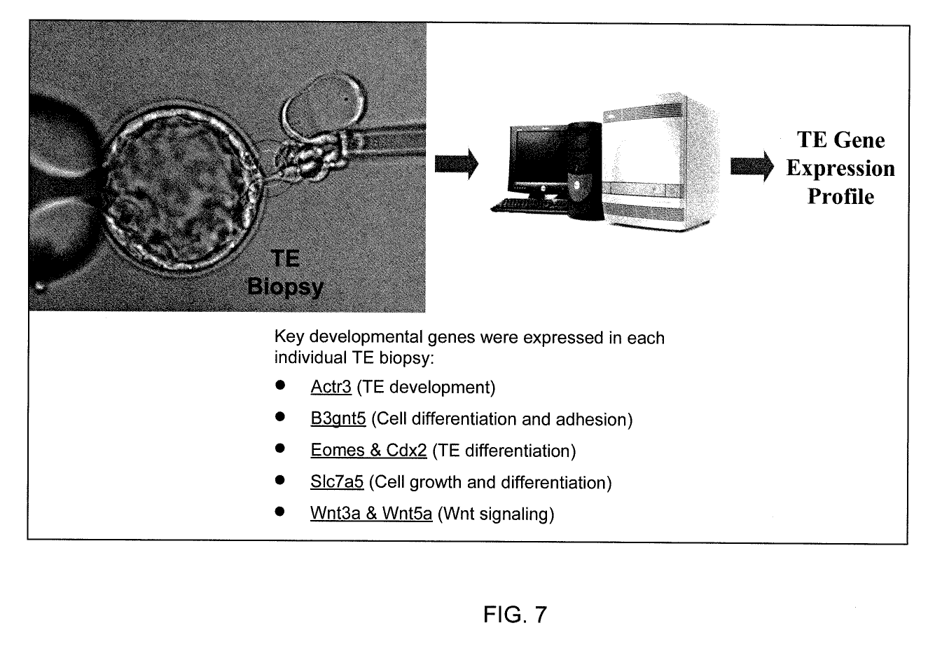

FIG. 7 displays certain developmental genes expressed in each individual murine TE biopsy as analyzed by quantitative real-time PCR relative to the housekeeping murine gene, Gapdh (GeneID: 14433). As shown in FIG. 7, examples of expressed developmental murine genes include Actr3 (GeneID: 74117), B3gnt5 (GeneID: 108105), Eomes (GeneID: 13813), Cdx2 (GeneID: 12591), Slc7a5 (GeneID: 20539), Wnt3a (GeneID: 22416) and Wnt5a (GeneID: 22418). In particular, the murine genes, B3gnt5 and Slc7a5, are involved in cell growth, cell differentiation and cell adhesion. The murine gene Actr3 is involved in TE development and the murine genes, Eomes and Cdx2, are involved in TE differentiation. Evaluating the expression of such murine genes using real-time PCR permits a molecular understanding of potential implantation success.

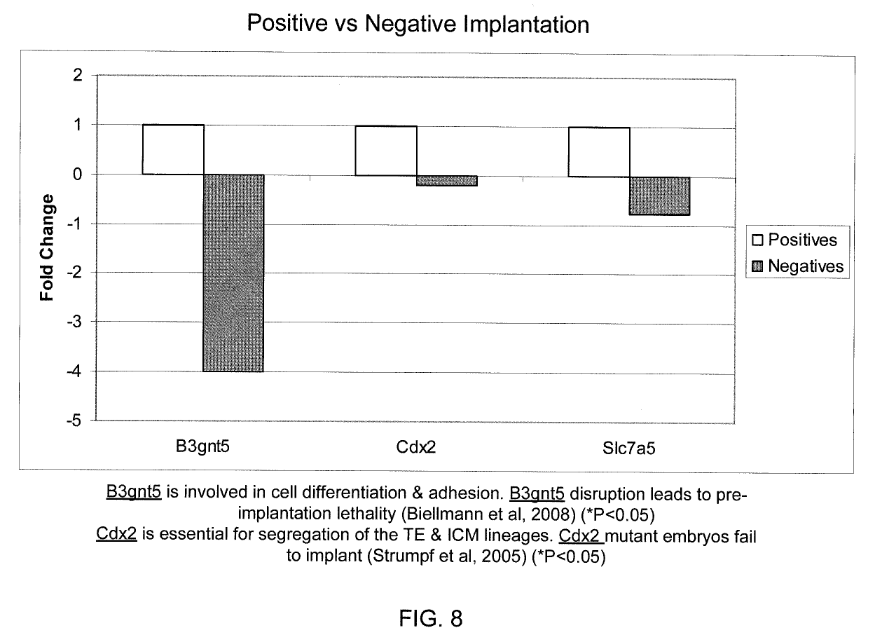

FIG. 8 displays the changes in gene expression for murine embryos that undergo viable implantation versus murine embryos that undergo non-viable implantation. As shown in FIG. 8, TE biopsies from murine blastocysts that failed to implant showed significant decrease in expression of the murine gene, B3gnt5. The murine gene B3gnt5 is involved in cell differentiation and adhesion. Studies indicate that disruptions in the murine gene B3gnt5 results in pre-implantation lethality. Additionally, murine blastocysts that failed to implant showed a decrease in expression of the murine gene, Cdx2. The murine gene, Cdx2, is a caudal-type homeodomain transcription factor and is expressed in TE at the blastocyst stage. In concordance with these results, mutant embryos of both of these genes result in pre-implantation lethality.

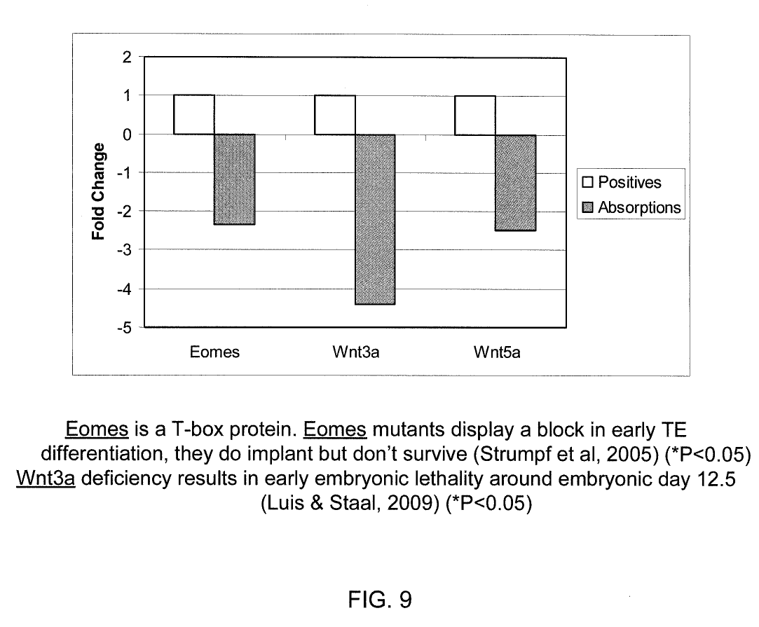

FIG. 9 displays the changes in gene expression for murine embryos that undergo viable implantation versus murine embryos that undergo non-viable absorption. The results demonstrate a significant decrease in expression of the murine gene, Eomes, for murine embryos that undergo non-viable absorption. The murine gene Eomes codes for the Eomesodermin T-Box Protein that is expressed in murine TE at the blastocyst stage and is further involved in TE differentiation. Moreover, the results in FIG. 9 illustrate a significant decrease in expression of the murine genes, Wnt3a and Wnt5a, for murine embryos that undergo non-viable absorption. The murine genes, Wnta3 and Wnt5a, are generally involved in the Wnt signaling pathway that involves hematopoiesis. In particular, studies showed that a deficiency in murine Wnt3a gene results in early embryonic lethality at approximately embryonic day 12.5.

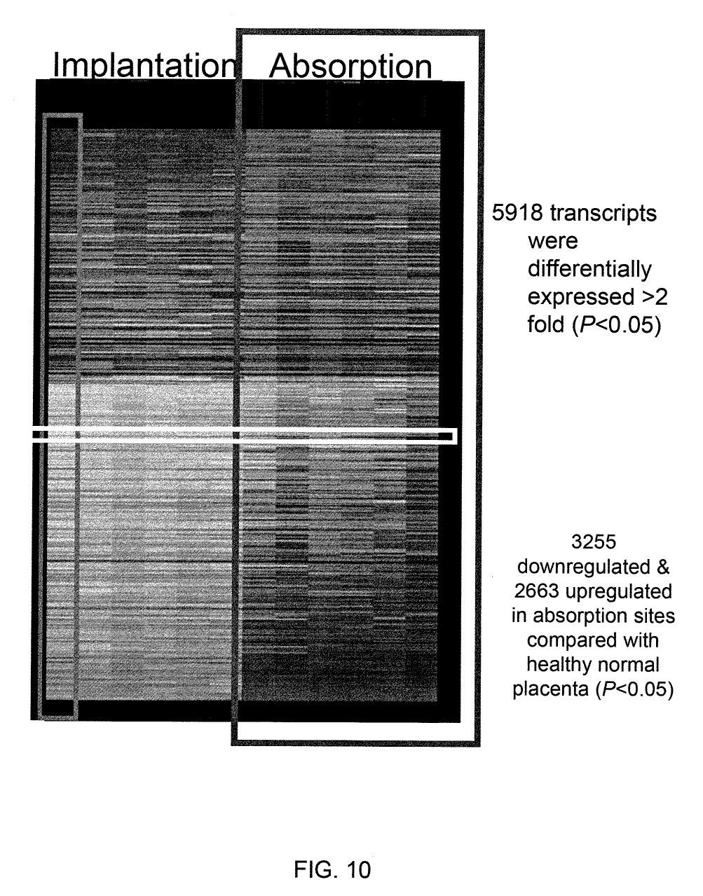

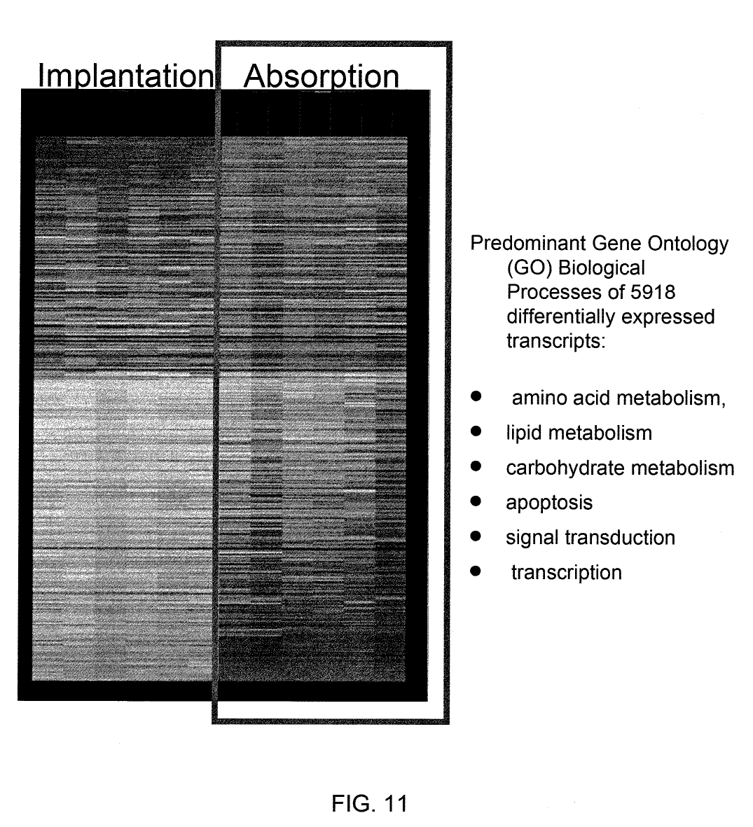

FIG. 10 displays whole transcriptome analysis of murine embryos that undergo viable implantation and murine embryos that undergo non-viable absorption. This whole transcriptome analysis reveals two distinct tissue types following microarray analysis with Genespring software. FIG. 10 displays an unsupervised hierarchical clustering of all 12 transcriptome microarrays. Each individual column is a single sample, and each row is a single transcript, with clear separation of the two tissue types. Expression is color coded with blue indicating low expression and red high expression. Almost six thousand (i.e., 5918) transcripts were differentially expressed with a >2 fold change (P<0.05). In particular, over three thousand (i.e., 3255) were downregulated and over two thousand (i.e., 2663) were upregulated in absorptions compared with healthy placental tissue.

FIG. 11 further illustrates the molecular differences between murine embryos that undergo viable implantation and murine embryos that undergo non-viable absorption using whole transcriptome analysis. FIG. 11 shows annotation of almost six thousand (i.e., 5918) differentially expressed transcripts. In particular, the predominant gene ontology (GO) biological processes of the 5918 differentially expressed genes include amino acid, lipid and carbohydrate metabolism, as well as apoptosis, signal transduction and transcription.

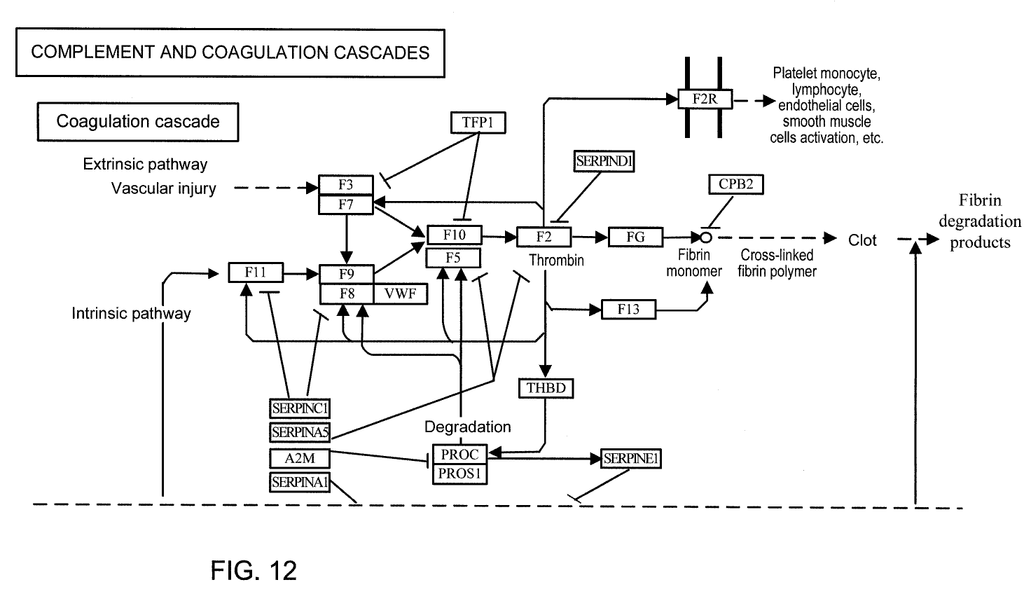

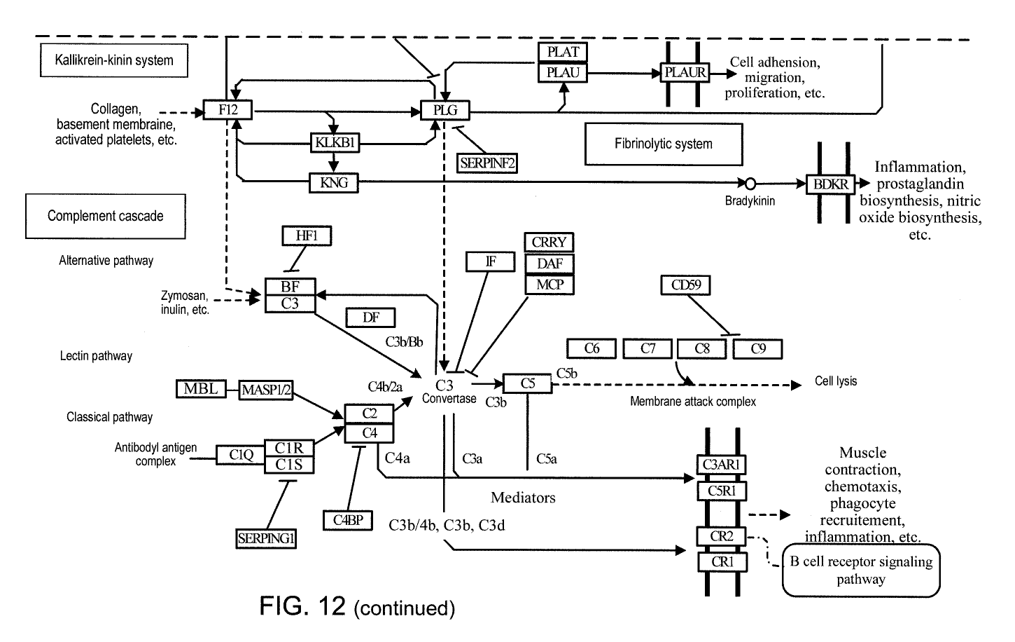

FIG. 12 shows that, in the absorption group, one of the predominant upregulated processes identified was the complement and coagulation cascade, in which twenty-six different transcripts were identified. Pregnancy is a pro-inflammatory/hypercoagulable state. Studies showed that complement activation plays an essential and causative role in pregnancy loss and fetal growth restriction, and that blocking activation of the complement cascade rescues pregnancies.

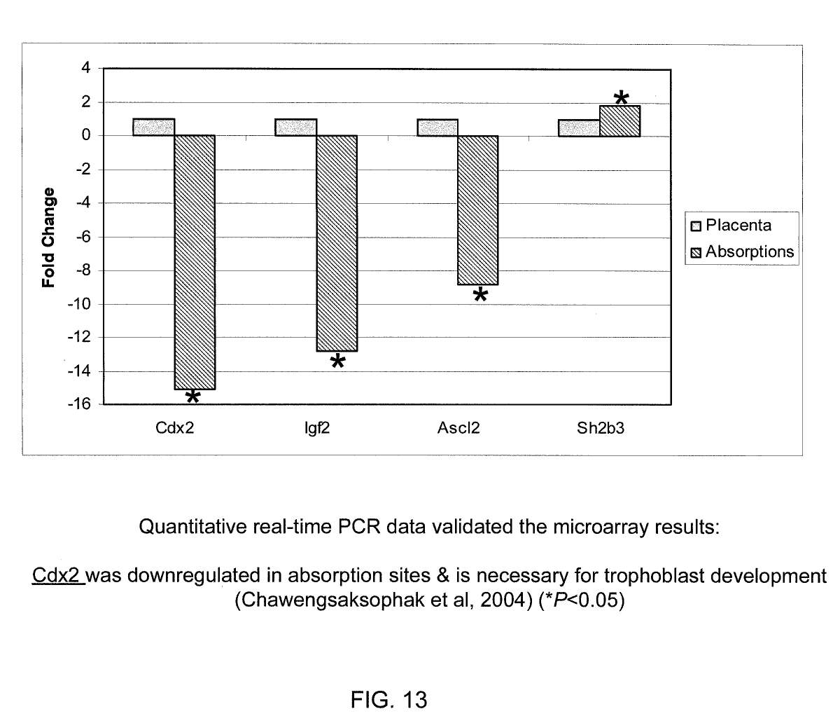

FIG. 13 displays the changes in gene expression for murine embryos that undergo viable placenta implantation versus murine embryos that undergo non-viable absorption. More specifically, FIG. 13 illustrates quantitative real-time PCR data for particular placental genes involved in the physiology of embryo implantation. For example, the murine genes, Cdx2 (GeneID: 12591), Igf2 (GeneID: 16002) and Ascl2 (GeneID: 17173) are downregulated for murine embryos that undergo non-viable absorption. Conversely, the murine gene Sh2b3 (GeneID: 16923) is upregulated for murine embryos that undergo non-viable absorption.

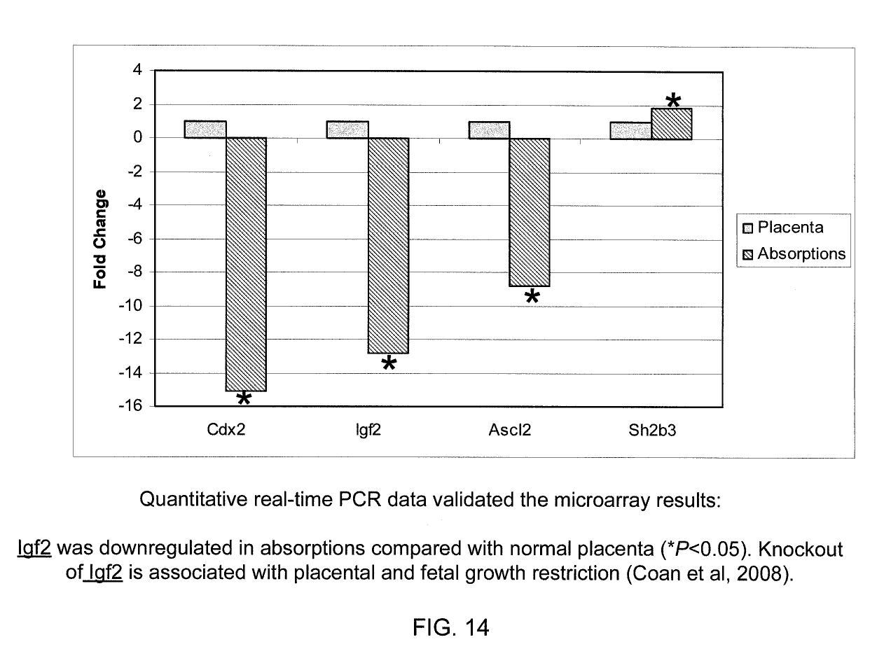

FIG. 14 shows the changes in gene expression for murine embryos that undergo viable placenta implantation versus murine embryos that undergo non-viable absorption as analyzed by real-time PCR. For example, FIG. 14 demonstrates that the murine gene Igf2 was downregulated in cases with non-viable absorptions as compared to cases with viable normal placenta (*P<0.05). The murine gene Igf2 is associated with placental and fetal growth restriction as illustrated by studies using knockout Igf2mice.

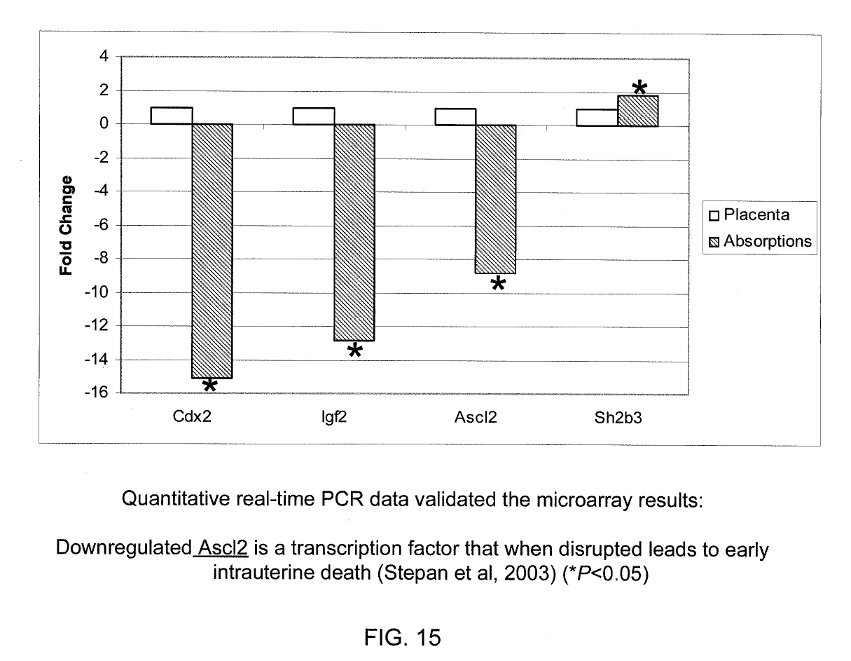

FIG. 15 shows the changes in gene expression for murine embryos that undergo viable placenta implantation versus murine embryos that undergo non-viable absorption as analyzed by real-time PCR. FIG. 15 demonstrates that the murine gene Ascl2 was downregulated in cases with non-viable absorption. The murine gene Ascl2 codes for a transcription factor that when disrupted leads to early intrauterine death.

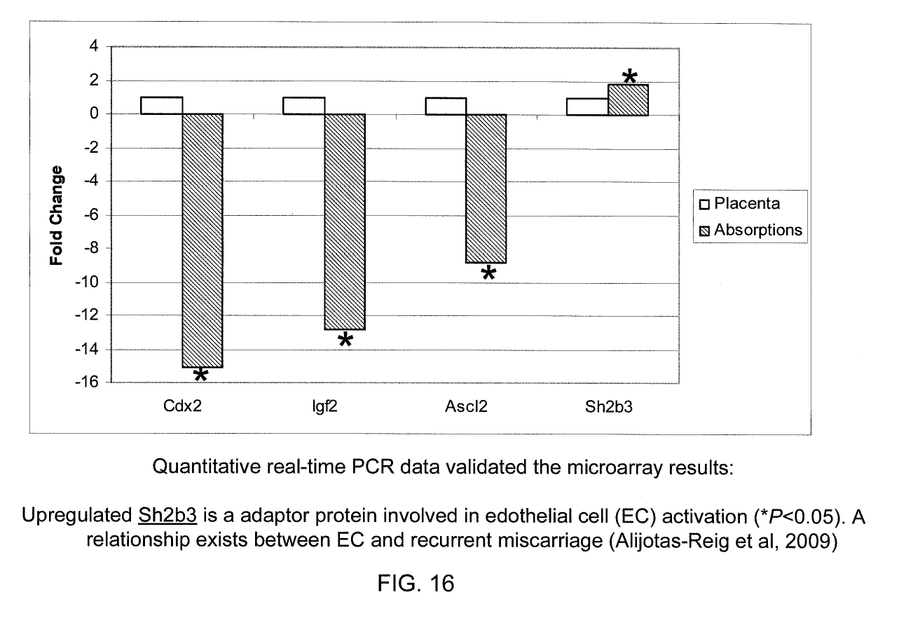

FIG. 16 shows the changes in gene expression for murine embryos that undergo viable placenta implantation versus murine embryos that undergo non-viable absorption as analyzed by real-time PCR. For example, FIG. 16 displays the upregulation of the murine gene Sh2b3 for cases with non-viable absorption. The murine gene Sh2b3 codes for an adaptor protein involved in endothelial cell (EC) activation. A relationship exists between EC activation and recurrent miscarriage.

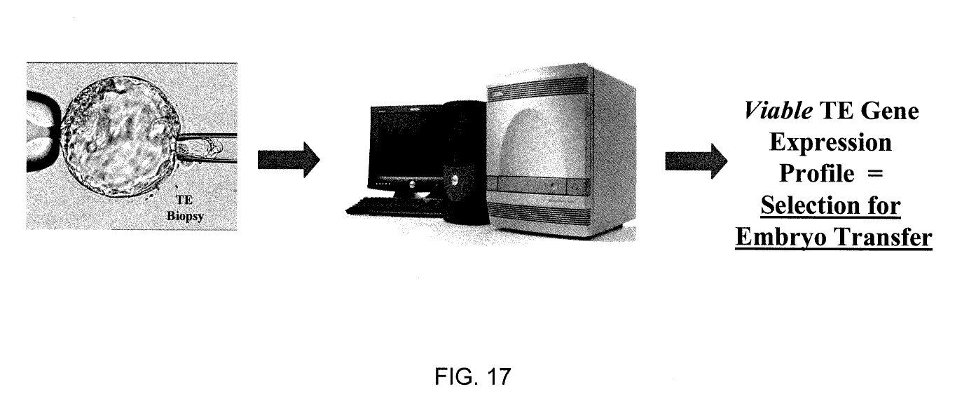

FIG. 17 illustrates significant features of the current invention, namely that an individual TE gene expression profile directly predicts ongoing healthy fetal development, in contrast to non-viable implantation (absorption), or complete implantation failure. Specifically, an important feature involves Wnt signaling, a signaling cascade which is crucial for embryonic development. TE transcriptome analysis may form the basis of quantifying blastocyst implantation potential by allowing for the identification of a viable TE expression profile. This, in combination with morphology, could be used for selection criteria prior to embryo transfer.

### Example 2

### Relating Gene Expression Profile to Implantation Success or Failure of a Human Embryo

Human cleavage-stage embryos are cultured in 10 μL drops of G1 supplemented with 2.5 mg/mL recombinant albumin under oil at 37° C., 6% CO₂, 5% O₂ for 24 hours. The embryos are washed twice in G2 culture media and further cultured in 10 μL drops of G2 supplemented with 2.5 mg/mL recombinant albumin under oil at 37° C., 6% CO₂, 5% O₂ for 48 hours with a fresh drop of G2 media added after 24 hours. At day 5, the human blastocyst TE cells are biopsied using a laser to obtain genetic material for gene expression profiling.

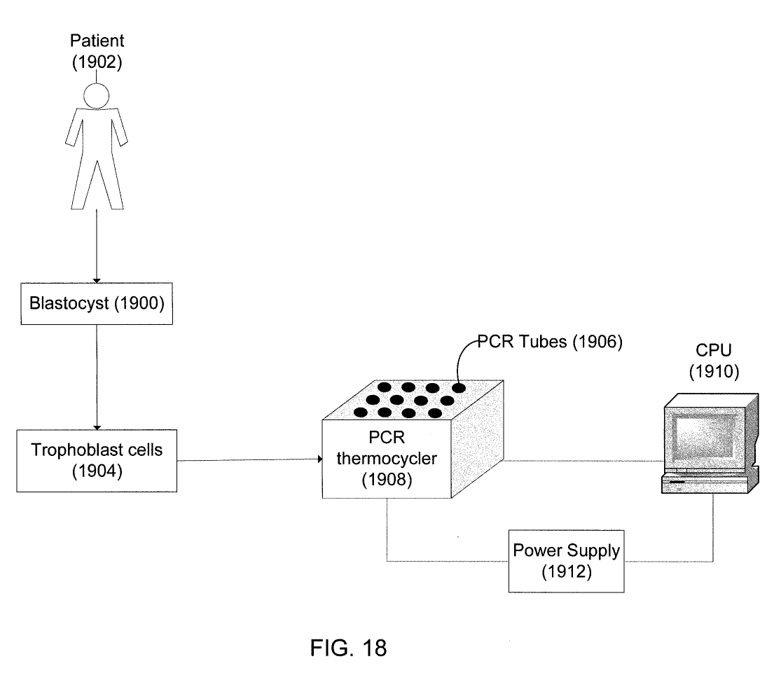

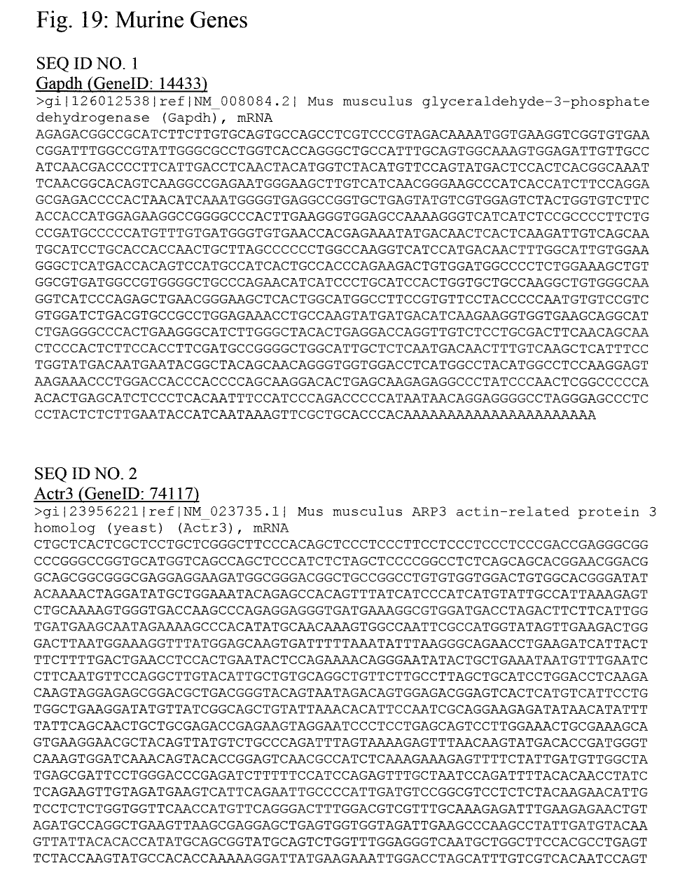

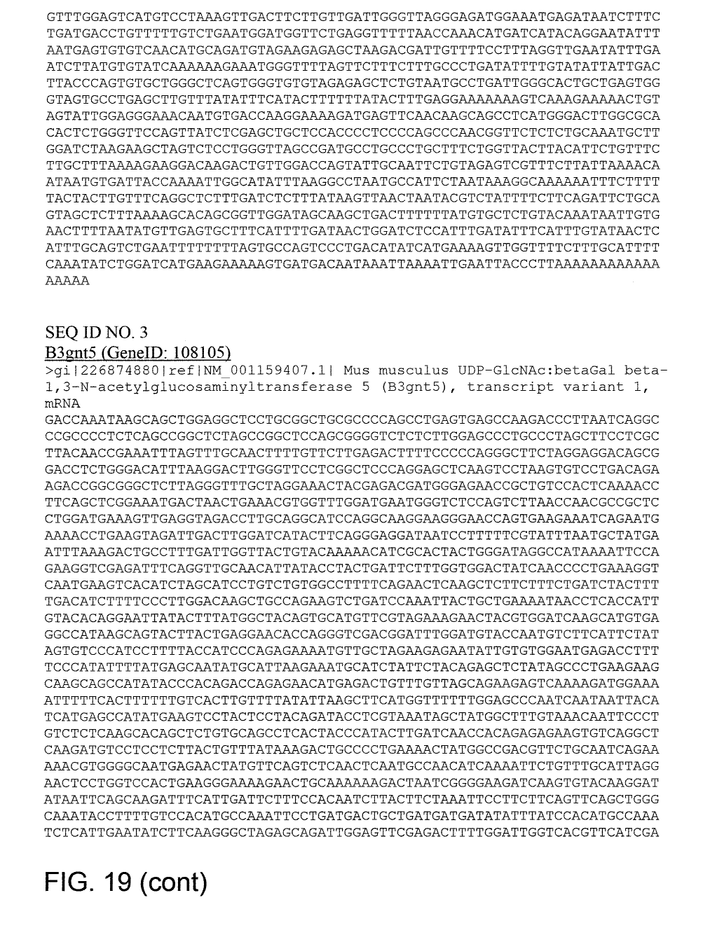

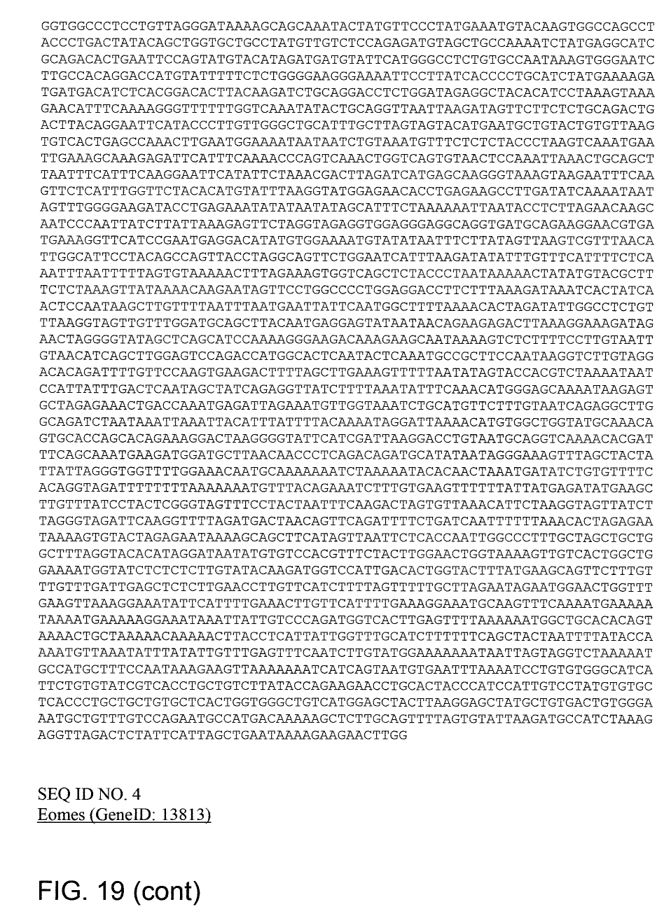

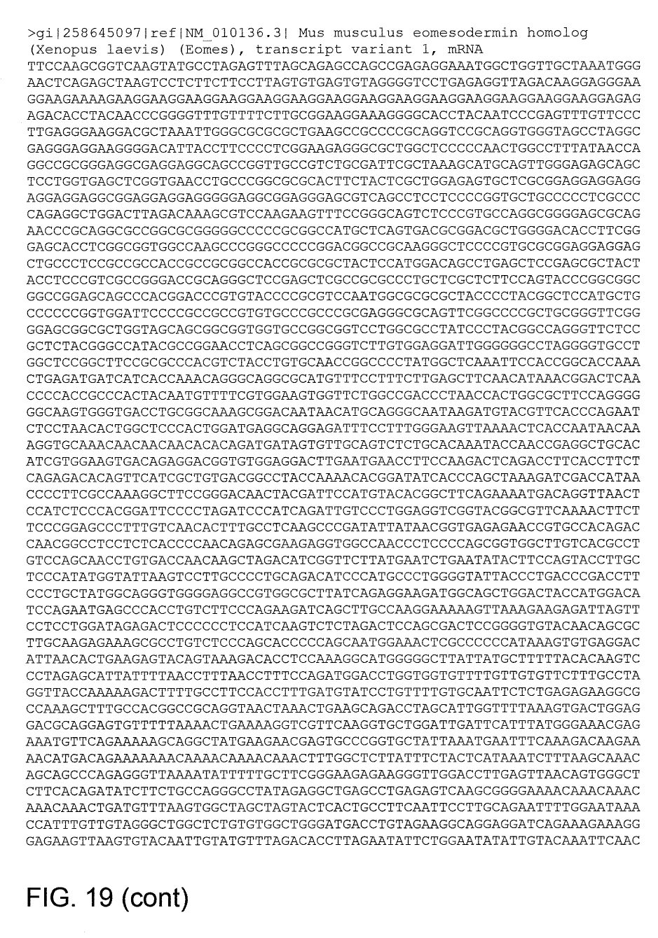

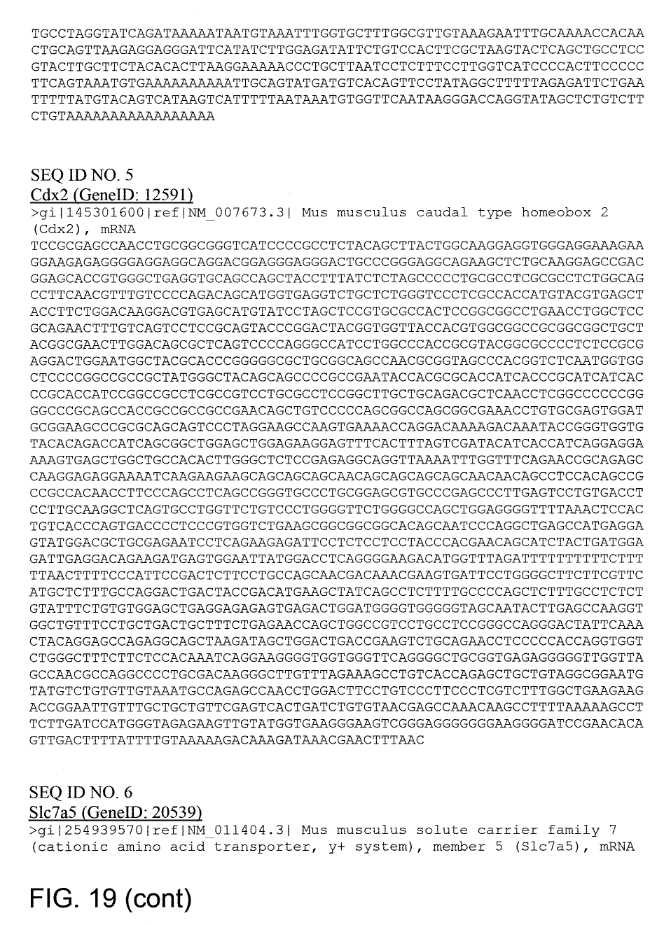

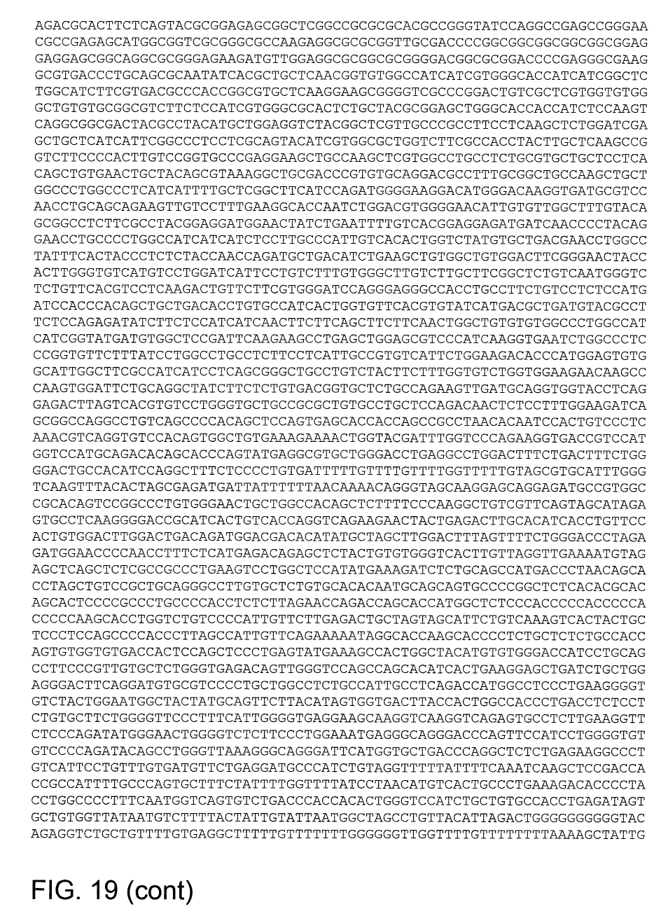

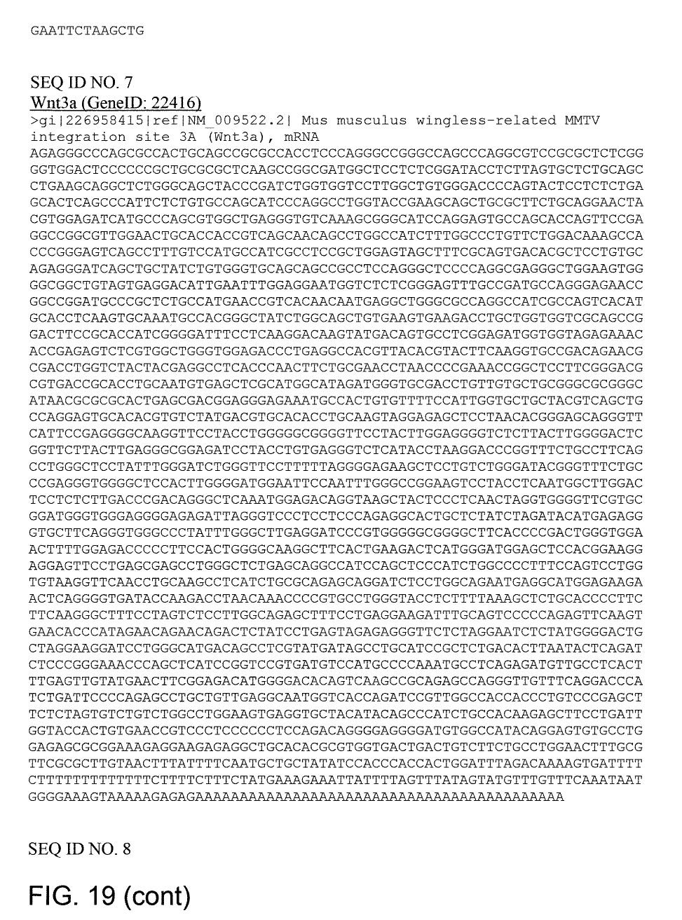

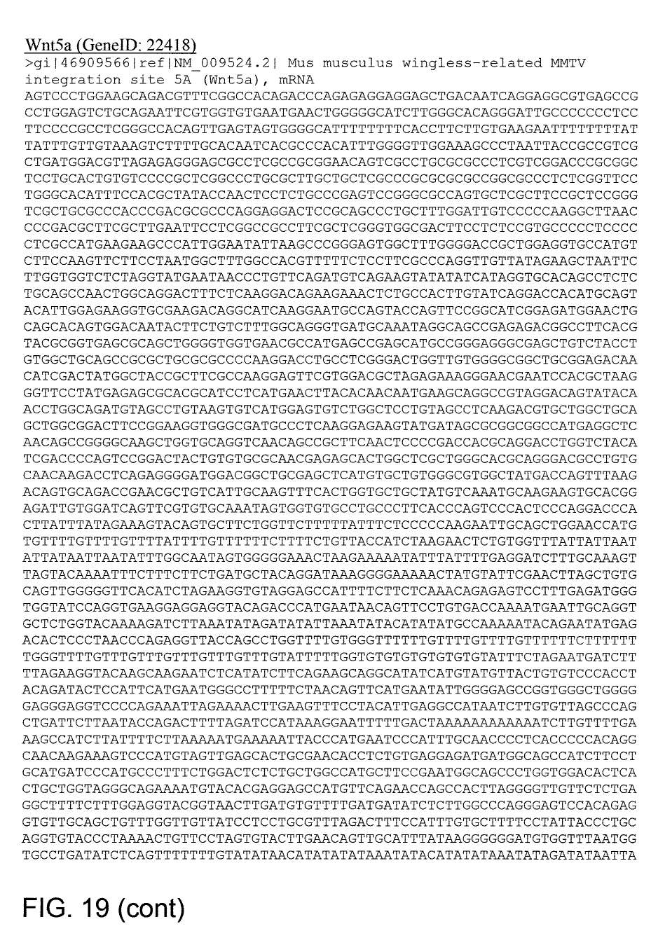

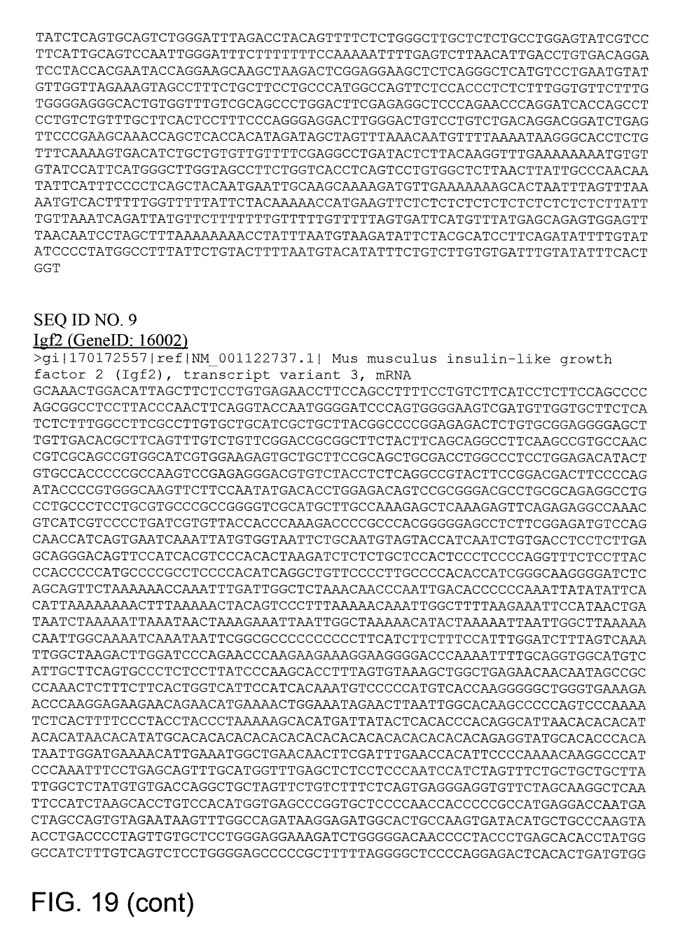

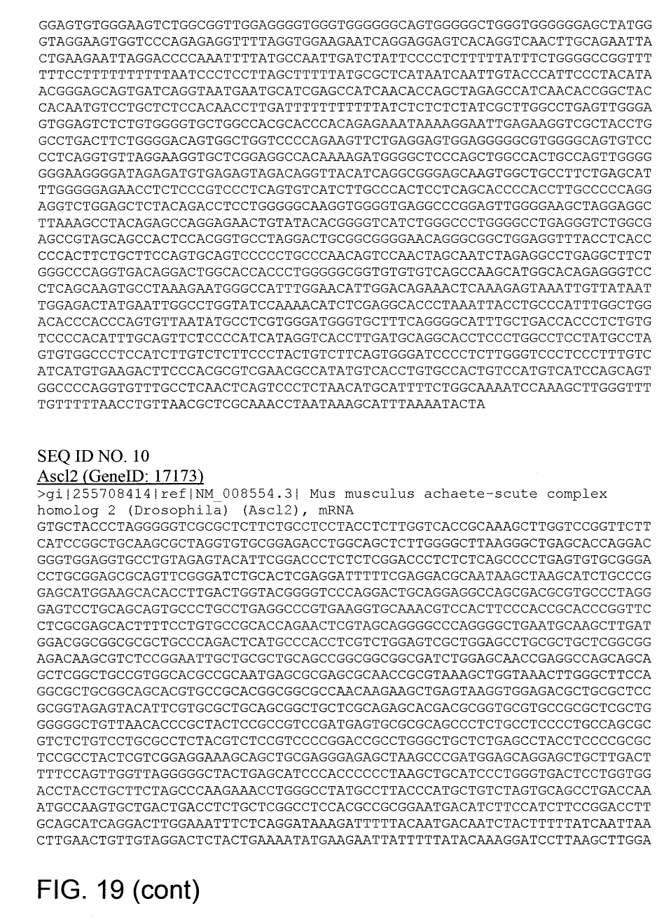

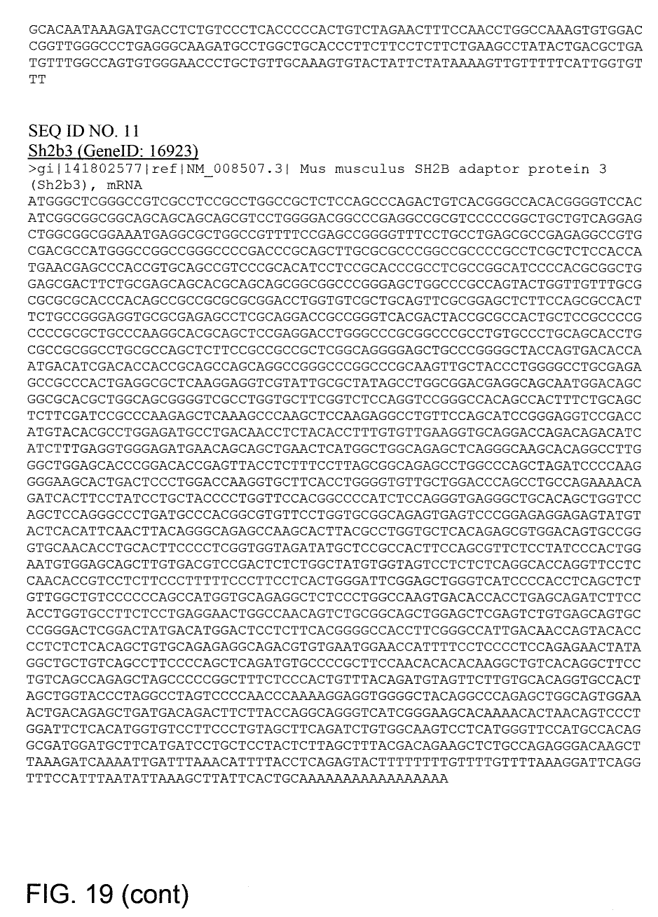

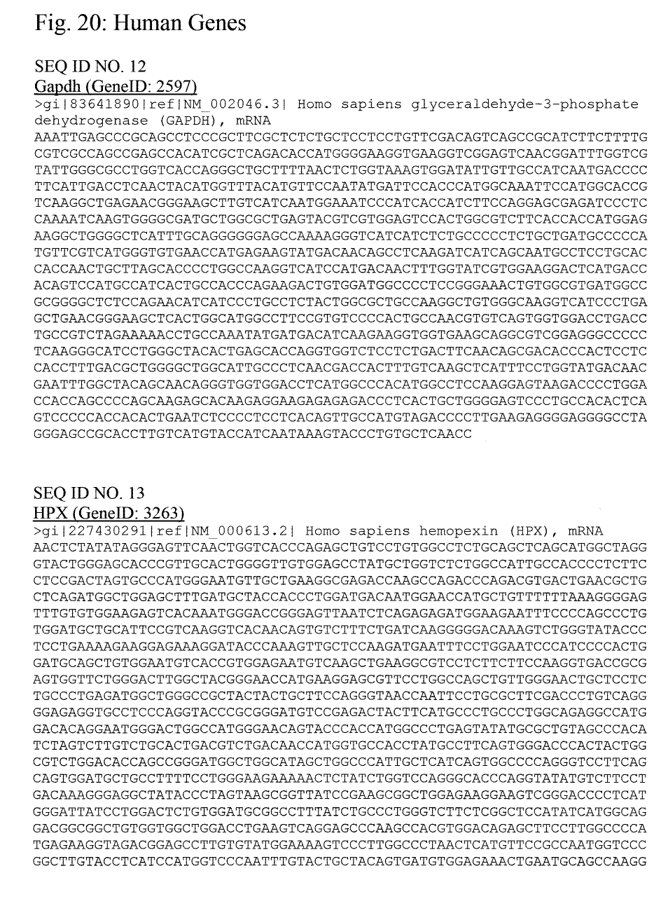

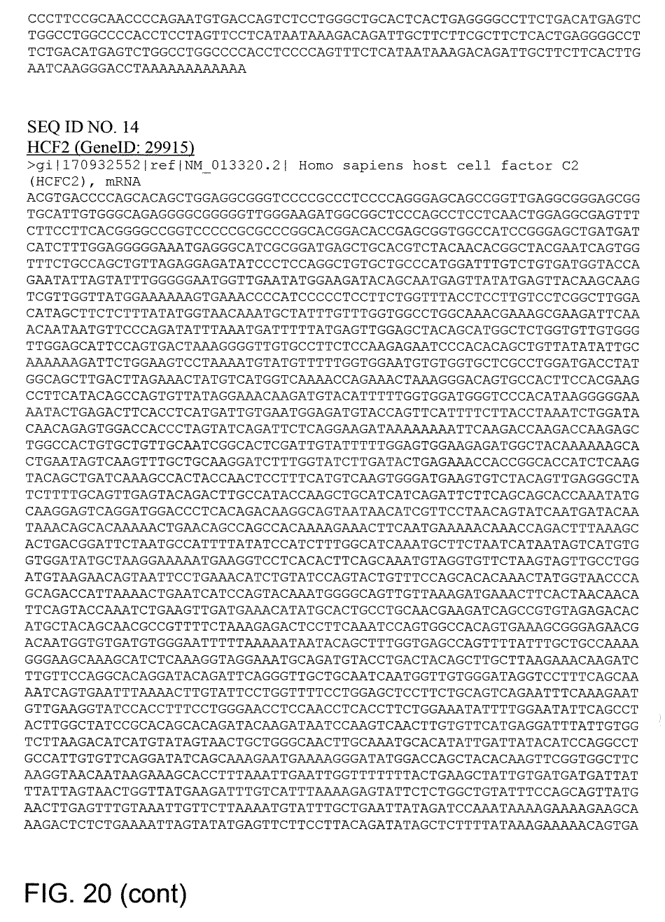

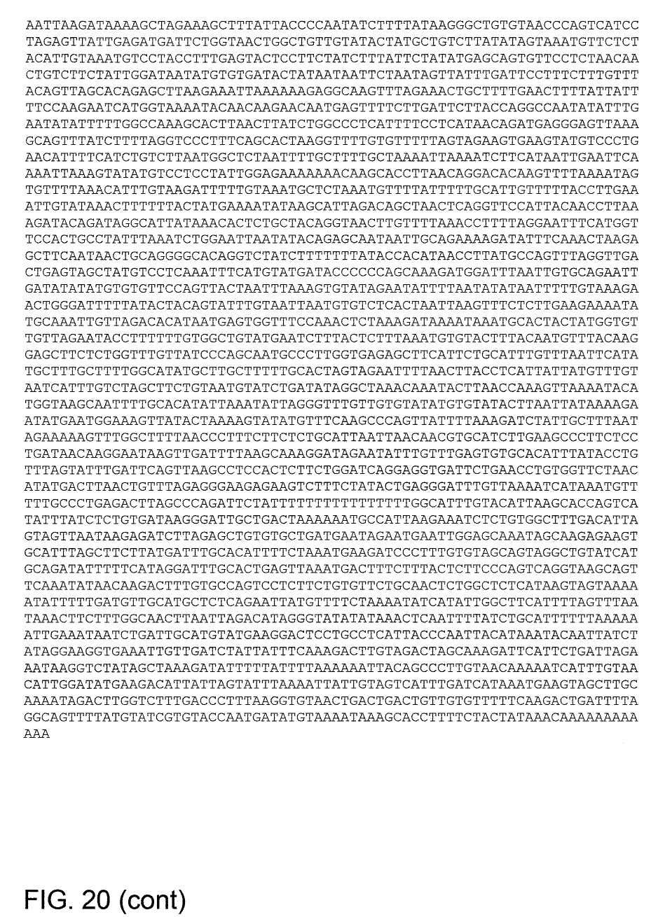

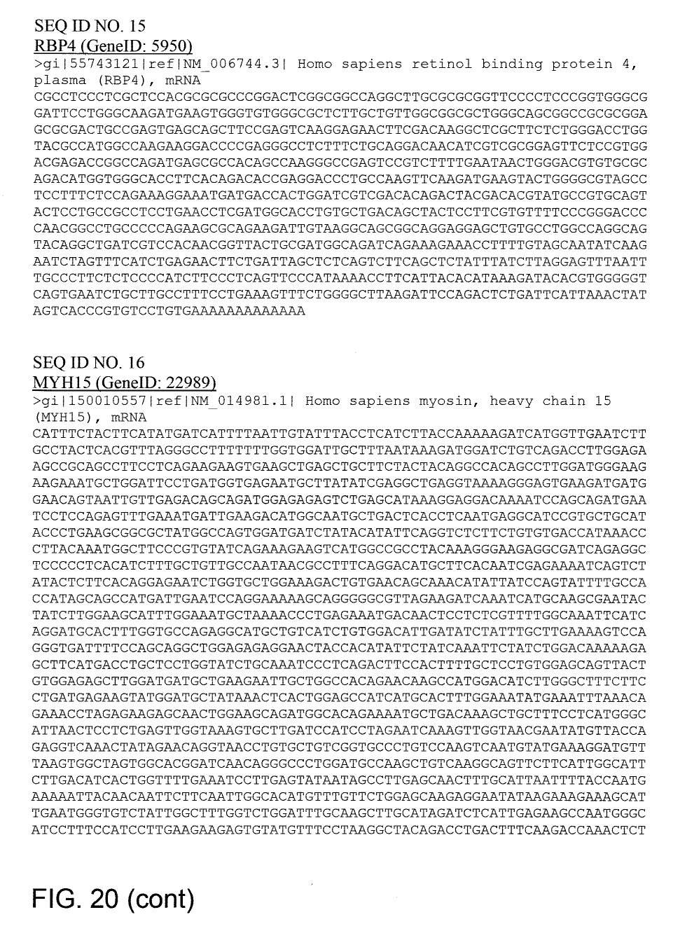

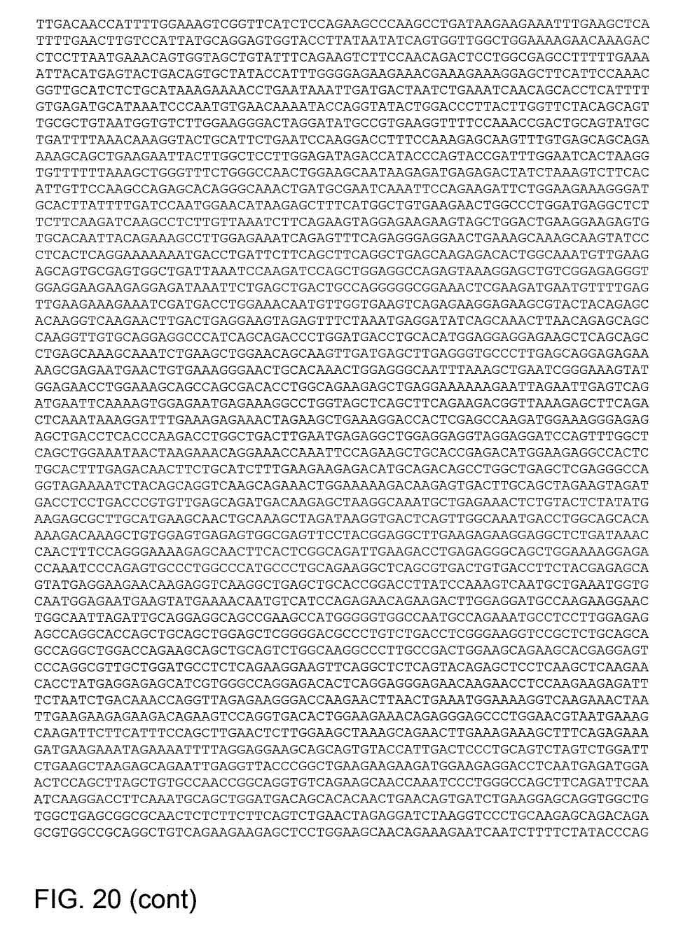

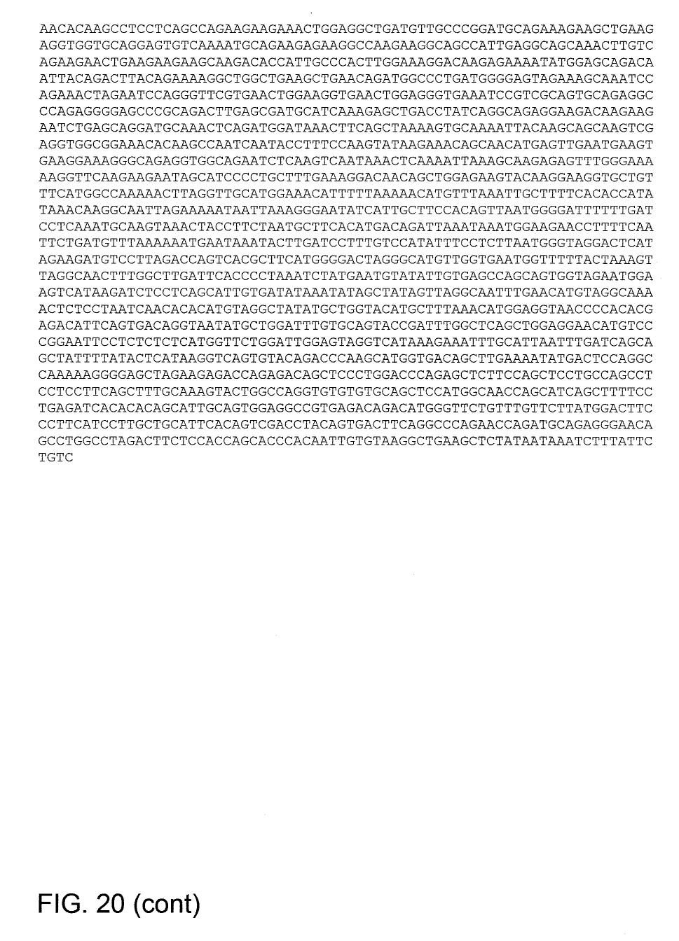

FIG. 18 illustrates a process for determining a gene expression profile to indicate the potential for implantation success of a blastocyst (1900). As shown in FIG. 18, a blastocyst (1900) originating from a fertilized egg of patient (1902) is biopsied for trophoblast cells (1904). The genetic material from the trophoblast cells (1904) is extracted and amplified using PCR. Individual primers corresponding to a panel of genes, each gene selected for significant developmental competence and implantation potential of the blastocyst (1900), are allocated into separate polypropylene PCR tubes (1906) atop a PCR thermocycler (1908). In one example, amplification of genetic material and analysis of gene expression is performed using a real time PCR thermocycler (Applied Biosystems, Foster City, Calif.). The PCR thermocycler (1908) is connected to a CPU (1910) and both PCR thermocycler (1908) and CPU (1910) are connected to power supply (1912). The CPU (1910) permits real-time monitoring of the gene expression pattern of the blastocyst (1900). In one embodiment, the increase in fluorescence signal from a Taqman® reporter probe indicates an increase of the gene product and permits the calculation of a cycle threshold (Ct) value. Analysis of the Ct values indicates a gene expression profile for each unique blastocyst that indicates potential for implantation success. For example, the up-regulation of certain human genes including HPX (GeneID: 3263), HCF2 (GeneID: 29915), RBP4 (GeneID: 5950) and MYH15 (GeneID: 22989) provides valuable molecular diagnostic information assessing the implantation potential of human embryos. Evaluation of these gene expression profiles provides valuable insight into the recommendation to successfully implant the blastocyst.

In one embodiment, developmental genes are monitored during the quantitative real-time PCR reaction using primers designed for each gene of interest. These developmental genes are analyzed relative to an endogenous housekeeping reference human gene, glyceraldehyde-3-p-dehydrogenase (Gapdh, GeneID: 2597). The human Gapdh gene also permits normalization between differing samples.

In another embodiment, gene expression analysis is performed using microarray technology. For example, transcriptome analysis is performed with Codelink™ Whole Genome Human Bioarrays (Applied Microarrays, Tempe, Ariz.) that contains over 57,000 transcripts using the manufacturer's recommended protocols. Array analysis using a microarray scanner reveals molecular profiles of significant genes implicated in implantation success. This array analysis can also be used to reveal molecular profiles of significant genes implicated in absorption of non-viable embryos by using isolated RNA from a placental tissue biopsy.

It will be appreciated that perceptive use of the instrumentalities described herein may result in a better selection of healthy blastocysts for implantation. Thus, fewer blastocysts need to be implemented, such that there is lower risk of multiple pregnancies while achieving a higher overall pregnancy success rate. The process described herein may be adapted as a molecular diagnostic tool for human use by identifying gene expression pattern of human blastocyst genes.

1. A system for increasing the probability of a successful pregnancy resulting from an in vitro fertilization, said system comprising:
means for determining the expression profile of at least one gene in a cell from a blastocyst resulting from said in vitro fertilization; and
means for determining the probability of success for the pregnancy resulting from said blastocyst.

2. The system of claim 1, further comprising genetic material obtained from said blastocyst.

3. The system of claim 1, further comprising means for determining whether or not to implant the blastocyst based on said determination of said probability of success of pregnancy.

4. The system of claim 1, further comprising means for recommending whether or not to implant the blastocyst based on said determination of said probability of success of pregnancy.

5. The system of claim 1, wherein said gene expression profile of said blastocyst is determined by quantitative polymerase chain reaction (PCR) or microarray analysis.

6. The system of claim 5, wherein said gene expression profile of said blastocyst is determined by quantitative real time PCR.

7. The system of claim 1, wherein said at least one gene is implicated in at least one function of implantation, absorption or development of said blastocyst.

8. The system of claim 1, wherein said at least one gene is selected from the group consisting of polynucleotide of SEQ ID Nos 2-11 and SEQ ID Nos 13-16.

9. A method for increasing the probability of a successful pregnancy resulting from an in vitro fertilization, said method comprising:
(a) determining the expression profile of at least one gene in a cell from a blastocyst resulting from said in vitro fertilization; and
(b) determining the probability of a successful pregnancy resulting from said blastocyst, said probability being determined based upon the expression profile of said at least one gene.

10. The method of claim 9, further comprising a step of determining whether to implant said blastocyst based on said probability of successful pregnancy determined in step (b).

11. The method of claim 9, further comprising a step of recommending whether to implant said blastocyst based on said probability of successful pregnancy determined in step (b).

12. The method of claim 9, wherein said expression profile of said blastocyst is determined by quantitative polymerase chain reaction (PCR) or microarray analysis.

13. The method of claim 9, wherein said at least one gene is implicated in at least one function of implantation, absorption or development of a blastocyst.

14. The method of claim 9, wherein said at least one gene is selected from the group consisting of polynucleotides of SEQ ID Nos 2-11 and SEQ ID Nos 13-16.

15. The method of claim 9, wherein said at least one gene is selected from the group consisting of polynucleotides of SEQ ID Nos 3-10, and wherein downregulation of said at least one gene as compared to a normalized control indicates a decreased probability of a successful pregnancy resulting from said blastocyst.

16. The method of claim 15, wherein the gene encoding glyceraldehyde-3-p-dehydrogenase (GAPDH) is used as said normalized control.

17. The method of claim 9, wherein said at least one gene is selected from the group consisting of polynucleotides of SEQ ID No. 11, and wherein upregulation of said at least one gene as compared to a normalized control indicates a decreased probability of a successful pregnancy.

18. The method of claim 17, wherein the gene encoding glyceraldehyde-3-p-dehydrogenase (GAPDH) is used as said normalized control.
